(19) United States
(12) Patent Application Publication
(10) Pub. No.: US 2026/0041449 A1
(43) Pub. Date: Feb. 12, 2026

(54) SURGICAL INSTRUMENT WITH ONE OR MORE FINGER-PRESSURE ACTIVATABLE POWER MODES

(71) Applicant: CILAG GMBH INTERNATIONAL, ZUG (CH)

(72) Inventor: JOSHUA MORGAN, CINCINNATI, OH (US)

(73) Assignee: CILAG GMBH INTERNATIONAL, ZUG (CH)

(21) Appl. No.: 18/799,558

(22) Filed: Aug. 9, 2024

(51) Int. Cl. A61B 17/32; A61B 18/00; A61B 18/14

(52) U.S. Cl. CPC A61B 17/320092; A61B 18/1445; A61B 2017/320075; A61B 2017/320094; A61B 2017/320095; A61B 2018/00601; A61B 2018/0063; A61B 2018/00702; A61B 2018/00773; A61B 2018/146

(57) ABSTRACT

A surgical system is herein disclosed that includes a surgical instrument and a generator. The surgical instrument includes a handle assembly that is grasped by one or more hands of an operator, a shaft assembly connected to the handle assembly, an arm, an end effector connected to the shaft assembly that interacts with tissue, and a sensor that senses a characteristic of the arm, from which an amount of pressure experienced by the arm while the end effector interacts with the tissue is determined. The generator is connected to the surgical instrument and delivers, to the end effector, a power profile dynamically selected from a plurality of power profiles based on the determined amount of pressure experienced by the arm.

### BACKGROUND

The present disclosure relates to surgical instruments and, in various arrangements, to surgical instruments with activatable power modes.

A variety of surgical instruments include an end effector having a blade element that vibrates at ultrasonic frequencies to cut and/or seal tissue (e.g., by denaturing proteins in tissue cells). These instruments include one or more piezoelectric elements that convert electrical power into ultrasonic vibrations, which are communicated along an acoustic waveguide to the blade element. The precision of cutting and coagulation may be controlled by the operator's technique and adjusting the power level, blade edge angle, tissue traction, and blade pressure.

Examples of ultrasonic surgical instruments include the HARMONIC ACE® Ultrasonic Shears, the HARMONIC WAVE® Ultrasonic Shears, the HARMONIC FOCUS® Ultrasonic Shears, and the HARMONIC SYNERGY® Ultrasonic Blades, all by Ethicon Endo-Surgery, Inc. of Cincinnati, Ohio. Further examples of such devices and related concepts are disclosed in U.S. Pat. No. 5,322,055, entitled “Clamp Coagulator/Cutting System for Ultrasonic Surgical Instruments,” issued Jun. 21, 1994, the disclosure of which is incorporated by reference herein; U.S. Pat. No. 5,873,873, entitled “Ultrasonic Clamp Coagulator Apparatus Having Improved Clamp Mechanism,” issued Feb. 23, 1999, the disclosure of which is incorporated by reference herein; U.S. Pat. No. 5,980,510, entitled “Ultrasonic Clamp Coagulator Apparatus Having Improved Clamp Arm Pivot Mount,” issued Nov. 9, 1999, the disclosure of which is incorporated by reference herein; U.S. Pat. No. 6,283,981, entitled “Method of Balancing Asymmetric Ultrasonic Surgical Blades,” issued Sep. 4, 2001, the disclosure of which is incorporated by reference herein; U.S. Pat. No. 6,309,400, entitled “Curved Ultrasonic Blade having a Trapezoidal Cross Section,” issued Oct. 30, 2001, the disclosure of which is incorporated by reference herein; U.S. Pat. No. 6,325,811, entitled “Blades with Functional Balance Asymmetries for use with Ultrasonic Surgical Instruments,” issued Dec. 4, 2001, the disclosure of which is incorporated by reference herein; U.S. Pat. No. 6,423,082, entitled “Ultrasonic Surgical Blade with Improved Cutting and Coagulation Features,” issued Jul. 23, 2002, the disclosure of which is incorporated by reference herein; U.S. Pat. No. 6,773,444, entitled “Blades with Functional Balance Asymmetries for Use with Ultrasonic Surgical Instruments,” issued Aug. 10, 2004, the disclosure of which is incorporated by reference herein; U.S. Pat. No. 6,783,524, entitled “Robotic Surgical Tool with Ultrasound Cauterizing and Cutting Instrument,” issued Aug. 31, 2004, the disclosure of which is incorporated by reference herein; U.S. Pat. No. 8,057,498, entitled “Ultrasonic Surgical Instrument Blades,” issued Nov. 15, 2011, the disclosure of which is incorporated by reference herein; U.S. Pat. No. 8,461,744, entitled “Rotating Transducer Mount for Ultrasonic Surgical Instruments,” issued Jun. 11, 2013, the disclosure of which is incorporated by reference herein; U.S. Pat. No. 8,591,536, entitled “Ultrasonic Surgical Instrument Blades,” issued Nov. 26, 2013, the disclosure of which is incorporated by reference herein; U.S. Pat. No. 8,623,027, entitled “Ergonomic Surgical Instruments,” issued Jan. 7, 2014, the disclosure of which is incorporated by reference herein; and U.S. Pat. No. 10,368,894, the disclosure of which is incorporated by reference herein.

Still further examples of ultrasonic surgical instruments are disclosed in U.S. Pub. No. 2006/0079874, entitled “Tissue Pad for Use with an Ultrasonic Surgical Instrument,” published Apr. 13, 2006, the disclosure of which is incorporated by reference herein; U.S. Pub. No. 2007/0191713, entitled “Ultrasonic Device for Cutting and Coagulating,” published Aug. 16, 2007, the disclosure of which is incorporated by reference herein; U.S. Pub. No. 2007/0282333, entitled “Ultrasonic Waveguide and Blade,” published Dec. 6, 2007, the disclosure of which is incorporated by reference herein; U.S. Pub. No. 2008/0200940, entitled “Ultrasonic Device for Cutting and Coagulating,” published Aug. 21, 2008, the disclosure of which is incorporated by reference herein; U.S. Pub. No. 2008/0234710, entitled “Ultrasonic Surgical Instruments,” published Sep. 25, 2008, the disclosure of which is incorporated by reference herein; and U.S. Pub. No. 2010/0069940, entitled “Ultrasonic Device for Fingertip Control,” published Mar. 18, 2010, the disclosure of which is incorporated by reference herein.

Some ultrasonic surgical instruments may include a cordless transducer such as that disclosed in U.S. Pub. No. 2012/0112687, entitled “Recharge System for Medical Devices,” published May 10, 2012, the disclosure of which is incorporated by reference herein; U.S. Pub. No. 2012/0116265, entitled “Surgical Instrument with Charging Devices,” published May 10, 2012, the disclosure of which is incorporated by reference herein; and/or U.S. Pat. App. No. 61/410,603, filed Nov. 5, 2010, entitled “Energy-Based Surgical Instruments,” the disclosure of which is incorporated by reference herein.

Additionally, some ultrasonic surgical instruments may include an articulating shaft section. Examples of such ultrasonic surgical instruments are disclosed in U.S. Pub. No. 2014/0005701, published Jan. 2, 2014, entitled “Surgical Instruments with Articulating Shafts,” the disclosure of which is incorporated by reference herein; and U.S. Pub. No. 2014/0114334, published Apr. 24, 2014, entitled “Flexible Harmonic Waveguides/Blades for Surgical Instruments,”the disclosure of which is incorporated by reference herein.

While several surgical instruments and systems have been made and used, it is believed that no one prior to the inventors has made or used the technology described in the appended claims.

### SUMMARY

The disclosed technology describes a surgical system. The surgical system comprises a surgical instrument and a generator. The surgical instrument comprises a handle assembly configured to be grasped by one or more hands of an operator, a shaft assembly connected to the handle assembly, an arm, an end effector connected to the shaft assembly and configured to interact with tissue, and a sensor configured to sense a characteristic of the arm, from which an amount of pressure experienced by the arm while the end effector interacts with the tissue is determined. The generator is connected to the surgical instrument and is configured to: (1) dynamically scale a power profile to deliver to the end effector from a plurality of power profiles, the power profile being selected based on the amount of pressure falling within one of a plurality of pressure ranges, (2) deliver a first power profile to the end effector in response to the amount of pressure falling within a first pressure range, (3) scale to and deliver a second power profile, less than the first power profile, to the end effector in response to the amount of pressure falling within a second pressure range, less than the first pressure range, for a first period of time.

The disclosed technology describes a surgical system comprising a surgical instrument and a generator. The surgical instrument comprises a handle assembly configured to be grasped by one or more hands of an operator, a shaft assembly connected to the handle assembly, an arm, an end effector connected to the shaft assembly and configured to interact with tissue, and a sensor configured to sense a characteristic of the arm, from which an amount of pressure experienced by the arm while the end effector interacts with the tissue is determined. The generator is connected to the surgical instrument and is configured to deliver, to the end effector, a power profile dynamically selected from a plurality of power profiles based on the determined amount of pressure experienced by the arm.

The disclosed technology describes a surgical instrument comprising a handle assembly configured to be grasped by one or more hands of an operator, a shaft assembly connected to the handle assembly, an arm, and an end effector connected to the shaft assembly and configured to interact with tissue in response to actuation of the arm. The surgical instrument is configured to deliver a dynamically scaled amount of power delivered to the end effector based on an amount of pressure experienced by the arm while the end effector interacts with the tissue. The amount of power is selected from a plurality of discrete power profiles. Each of the discrete power profiles is selected based on the amount of pressure falling within one of a plurality of pressure ranges. Each pressure range of the plurality of pressure ranges has a predetermined range of pressures therein, and each power profile of the discrete power profiles maintains a uniform power until the amount of power delivered to the end effector is rescaled.

The disclosed technology describes a generator comprising a control circuit configured to: (1) receive a first signal from a surgical instrument, wherein the first signal comprises an amount of pressure experienced by an arm of a surgical instrument while the surgical instrument interacts with a tissue, (2) dynamically select, from a plurality of discrete power profiles, and based on the determined amount of pressure experienced by the arm, a first power profile, wherein each of the discrete power profiles is selected based on the amount of pressure falling within one of a plurality of pressure ranges, with each pressure range of the plurality of pressure ranges having a predetermined range of pressures therein, and (3) deliver, via an energy circuit, the first power profile of the plurality of profiles to the surgical instrument.

The disclosed technology describes a surgical system comprising a surgical instrument and a generator. The surgical instrument comprises a handle assembly configured to be grasped by one or more hands of an operator, a shaft assembly connected to the handle assembly and comprising an arm, an end effector connected to the shaft assembly and configured to interact with tissue, and a sensor configured to sense a characteristic of the arm, from which an amount of pressure experienced by the arm while the end effector interacts with the tissue is determined. The generator is connected to the surgical instrument and is configured to: (1) monitor the amount of pressure over time, (2) in response to the amount of pressure exceeding a first pressure threshold, deliver a power profile to the end effector at a first time, (3) determine whether a predetermined period of time has elapsed since the first time, (4) in response to the amount of pressure falling below the first pressure threshold before the predetermined period of time has elapsed, stop delivery of the power profile to the end effector, and in response to the amount of pressure exceeding the first pressure threshold for the predetermined period of time: (5) determine whether the amount of pressure exceeds a minimum threshold, in response to the amount of pressure exceeding the minimum threshold, (6) continue delivery of the power profile to the end effector, and in response to the amount of pressure falling below the minimum threshold, (7) stop delivery of the power profile to the end effector.

### BRIEF DESCRIPTION OF THE DRAWINGS

FIG. 1 depicts a block schematic view of an exemplary surgical system, in accordance with the disclosed technology;

FIG. 2 depicts a side elevational view of an exemplary form that an instrument of the system of FIG. 1 may take, in accordance with the disclosed technology;

FIG. 3 depicts a side elevational view of an exemplary alternative form that an instrument of the system of FIG. 1 may take, in accordance with the disclosed technology;

FIG. 4A depicts a cross-sectional view of the instrument of FIG. 3, taken along line 4-4 of FIG. 3, showing an end effector of the instrument in a first partially closed clamping configuration, in accordance with the disclosed technology;

FIG. 4B depicts a cross-sectional view of the instrument of FIG. 3, taken along line 4-4 of FIG. 3, showing the end effector in a second partially closed clamping configuration, in accordance with the disclosed technology, in accordance with the disclosed technology;

FIG. 4C depicts a cross-sectional view of the instrument of FIG. 3, taken along line 4-4 of FIG. 3, showing the end effector in a fully closed clamping configuration, in accordance with the disclosed technology, in accordance with the disclosed technology;

FIG. 5 depicts a cross-sectional view of the instrument of FIG. 3, taken along line 5-5 of FIG. 3, in accordance with the disclosed technology;

FIG. 6 depicts a side elevational view of an exemplary alternative form that an instrument of the system of FIG. 1 may take, in accordance with the disclosed technology;

FIG. 7 depicts a side elevational view of an exemplary alternative form that an instrument of the system of FIG. 1 may take, in accordance with the disclosed technology;

FIG. 8A depicts a graph of finger pressure and generator power profiles over time using an instrument of the system of FIG. 1, in accordance with the disclosed technology;

FIG. 8B depicts another graph of finger pressure and generator power profiles over time using an instrument of the system of FIG. 1, in accordance with the disclosed technology;

FIG. 9 depicts a logic flow diagram for selecting a power profile corresponding to a tissue algorithm that may be implemented by a generator, in accordance with the disclosed technology;

FIG. 10 depicts another graph of finger pressure and generator power profiles over time using an instrument of the system of FIG. 1, in accordance with the disclosed technology; and

FIG. 11 depicts a logic flow diagram for selecting a power profile, from a plurality of power profiles, corresponding to a tissue algorithm that may be implemented by a generator, in accordance with the disclosed technology.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a surgical system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to an operator operating a surgical instrument having a distal surgical end effector. The term “proximal” refers to the position of an element closer to the operator and the term “distal” refers to the position of an element closer to the surgical end effector and further away from the operator. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Furthermore, the use of “couple”, “coupled”, or similar phrases should not be construed as being limited to a certain number of components or a particular order of components unless the context clearly dictates otherwise.

Additionally, the terms “power profile” and “surgical mode” are oftentimes used interchangeably. It is noted that each surgical mode in the present system has a corresponding power profile (e.g., power level, which can stay constant or vary over the course of the surgical mode cycle).

### I. Overview of Exemplary Ultrasonic Surgical System

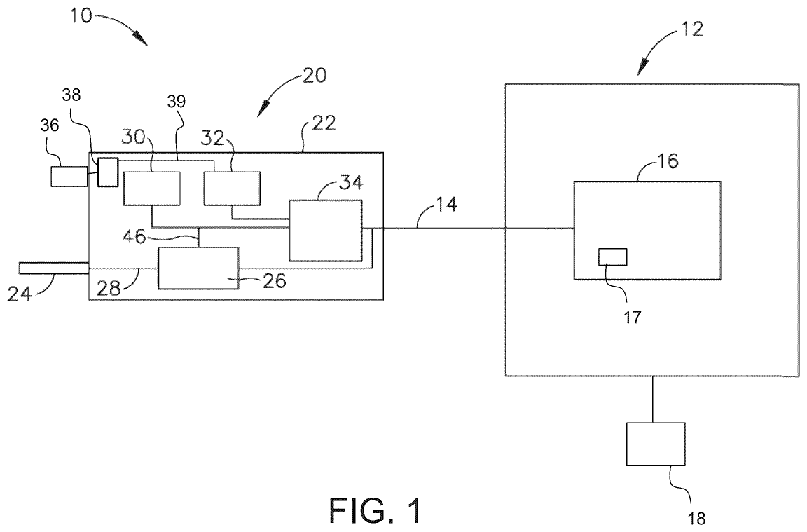

FIG. 1 shows components of an exemplary surgical system 10 in diagrammatic block form. As shown, system 10 comprises an ultrasonic generator 12 and an ultrasonic surgical instrument 20. As will be described in greater detail below, instrument 20 is operable to cut tissue and seal or weld tissue (e.g., a blood vessel, etc.) substantially simultaneously, using ultrasonic vibrational energy. Generator 12 and instrument 20 are coupled together via cable 14. Cable 14 may comprise a plurality of wires; and may provide unidirectional electrical communication from generator 12 to instrument 20 and/or bidirectional electrical communication between generator 12 and instrument 20. By way of example only, cable 14 may comprise a “hot” wire for electrical power to surgical instrument 20, a ground wire, and a signal wire for transmitting signals from surgical instrument 20 to ultrasonic generator 12, with a shield surrounding the three wires. In some versions, separate “hot” wires are used for separate activation voltages (e.g., one “hot” wire for a first activation voltage and another “hot” wire for a second activation voltage, or a variable voltage between the wires proportional to the power requested, etc.). Of course, any other suitable number or configuration of wires may be used. It should also be understood that some versions of system 10 may incorporate generator 12 into instrument 20, such that cable 14 may simply be omitted. Moreover, an output indicator 18 may be connected to the generator 12 and/or the surgical instrument to provide feedback to the operator.

By way of example only, generator 12 may comprise the GEN04, GEN11, or GEN 300 sold by Ethicon Endo-Surgery, Inc. of Cincinnati, Ohio. In addition or in the alternative, generator 12 may be constructed in accordance with at least some of the teachings of U.S. Pub. No. 2011/0087212, entitled “Surgical Generator for Ultrasonic and Electrosurgical Devices,” published Apr. 14, 2011, the disclosure of which is incorporated by reference herein. Alternatively, any other suitable generator 12 may be used. As will be described in greater detail below, generator 12 is operable to provide power to instrument 20 to perform ultrasonic surgical procedures.

Instrument 20 comprises a handpiece 22, which is configured to be grasped in one hand (or two hands) of an operator and manipulated by one hand (or two hands) of the operator during a surgical procedure. For instance, in some versions, handpiece 22 may be grasped like a pencil by the operator. In some other versions, handpiece 22 may include a scissor grip that may be grasped like scissors by the operator. In some other versions, handpiece 22 may include a pistol grip that may be grasped like a pistol by the operator. Of course, handpiece 22 may be configured to be gripped in any other suitable fashion. Furthermore, some versions of instrument 20 may substitute handpiece 22 with a body that is coupled to a robotic surgical system that is configured to operate instrument (e.g., via remote control, etc.). In the present example, a blade 24 extends distally from the handpiece 22. In some examples, a distal end of the blade 24 forms part of an end effector including the blade 24 and a clamp arm (described in greater detail below).

Handpiece 22 includes an ultrasonic transducer 26 and an ultrasonic waveguide 28, which couples ultrasonic transducer 26 with blade 24. Ultrasonic transducer 26 receives electrical power/signals from generator 12 via cable 14. By virtue of its piezoelectric properties, ultrasonic transducer 26 is operable to convert such electrical power into ultrasonic vibrational energy. Also, in the same or similar manner as described above with respect to instrument 20, the instrument 20 may be configurable to also provide radiofrequency (RF) energy (e.g., bipolar RF energy/power) to tissue in addition to or instead of ultrasonic energy. Various ways in which instrument may be configured and operable to provide both ultrasonic and RF electrosurgical modes of operation are either described in various references cited herein or will be apparent to those skilled in the art in view of the disclosure herein. As will be appreciated by those skilled in the art, the technology disclosed herein may be applied to harmonic instruments, bipolar RF instruments, advanced bipolar RF instruments, combined modalities instruments, and the like.

Ultrasonic waveguide 28 may be flexible, semi-flexible, rigid, or have any other suitable properties. As noted above, ultrasonic transducer 26 is integrally coupled with blade 24 via ultrasonic waveguide 28. In particular, when ultrasonic transducer 26 is activated to vibrate at ultrasonic frequencies, such vibrations are communicated through ultrasonic waveguide 28 to blade 24, such that blade 24 will also vibrate at ultrasonic frequencies. When blade 24 is in an activated state (i.e., vibrating ultrasonically), blade 24 is operable to effectively cut through tissue and seal tissue. Ultrasonic transducer 26, ultrasonic waveguide 28, and blade 24 together thus form an acoustic assembly providing ultrasonic energy for surgical procedures when powered by generator 12. Handpiece 22 is configured to substantially isolate the operator from the vibrations of the acoustic assembly formed by transducer 26, ultrasonic waveguide 28, and blade 24.

In some versions, ultrasonic waveguide 28 may amplify the mechanical vibrations transmitted through ultrasonic waveguide 28 to blade 24. Ultrasonic waveguide 28 may further have features to control the gain of the longitudinal vibration along ultrasonic waveguide 28 and/or features to tune ultrasonic waveguide 28 to the resonant frequency of system 10. For instance, ultrasonic waveguide 28 may have any suitable cross-sectional dimensions/configurations, such as a substantially uniform cross-section, be tapered at various sections, be tapered along its entire length, or have any other suitable configuration. Ultrasonic waveguide 28 may, for example, have a length substantially equal to an integral number of one-half system wavelengths (nλ/2). Ultrasonic waveguide 28 and blade 24 may be fabricated from a solid core shaft constructed out of a material or combination of materials that propagates ultrasonic energy efficiently, such as a titanium alloy (e.g., Ti-6Al-4V), aluminum alloys, sapphire, stainless steel, or any other acoustically compatible material or combination of materials.

In the present example, the distal end of blade 24 is located at a position corresponding to an anti-node associated with resonant ultrasonic vibrations communicated through waveguide 28 (i.e., at an acoustic anti-node), in order to tune the acoustic assembly to a preferred resonant frequency fₒ when the acoustic assembly is not loaded by tissue. When transducer 26 is energized, the distal end of blade 24 is configured to move longitudinally in the range of, for example, approximately 10 to 500 microns peak-to-peak, and in some instances in the range of about 20 to about 200 microns at a predetermined vibratory frequency fₒ of, for example, 55.5 kHz. When transducer 26 of the present example is activated, these mechanical oscillations are transmitted through waveguide 28 to reach blade 24, thereby providing oscillation of blade 24 at the resonant ultrasonic frequency. Thus, the ultrasonic oscillation of blade 24 may simultaneously sever the tissue and denature the proteins in adjacent tissue cells, thereby providing a coagulative effect with relatively little thermal spread. In some versions, an electrical current may also be provided through blade 24 to also cauterize the tissue.

By way of example only, ultrasonic waveguide 28 and blade 24 may comprise components sold by Ethicon Endo-Surgery, Inc. of Cincinnati, Ohio. By way of further example only, ultrasonic waveguide 28 and/or blade 24 may be constructed and operable in accordance with the disclosure of U.S. Pat. No. 6,423,082, entitled “Ultrasonic Surgical Blade with Improved Cutting and Coagulation Features,” issued Jul. 23, 2002, the disclosure of which is incorporated by reference herein. As another merely illustrative example, ultrasonic waveguide 28 and/or blade 24 may be constructed and operable in accordance with the teachings of U.S. Pat. No. 5,324,299, entitled “Ultrasonic Scalpel Blade and Methods of Application,” issued Jun. 28, 1994, the disclosure of which is incorporated by reference herein. Other suitable properties and configurations of ultrasonic waveguide 28 and blade 24 will be apparent to those skilled in the art in view of the teachings herein.

Handpiece 22 of the present example also includes a control selector 30 and an activation switch 32, which are each in communication with a circuit board 34. By way of example only, circuit board 34 may comprise a conventional printed circuit board, a flex circuit, a rigid-flex circuit, or may have any other suitable configuration. Control selector 30 and activation switch 32 may be in communication with circuit board 34 via one or more wires, traces formed in a circuit board or flex circuit, and/or in any other suitable fashion. Circuit board 34 is coupled with cable 14, which is in turn coupled with control circuitry 16 within generator 12. Activation switch 32 is operable to selectively activate power to ultrasonic transducer 26. In particular, when switch 32 is activated, such activation provides communication of appropriate power to ultrasonic transducer 26 via cable 14. By way of example only, activation switch 32 may be constructed in accordance with any of the teachings of the various references cited herein. Other various forms that activation switch 32 may take will be apparent to those skilled in the art in view of the teachings herein.

In the present example, surgical system 10 is operable to provide at least two different levels or types of ultrasonic energy (e.g., different frequencies and/or amplitudes, etc.) at blade 24. To that end, control selector 30 is operable to permit the operator to select a desired level/amplitude of ultrasonic energy. By way of example only, control selector 30 may be constructed in accordance with any of the teachings of the various references cited herein. Other various forms that control selector 30 may take will be apparent to those skilled in the art in view of the teachings herein. In some versions, when an operator makes a selection through control selector 30, the operator's selection is communicated back to control circuitry 16 of generator 12 via cable 14, and control circuitry 16 adjusts the power communicated from generator 12 accordingly the next time the operator actuates activation switch 32.

It should be understood that the level/amplitude of ultrasonic energy provided at blade 24 may be a function of characteristics of the electrical power communicated from generator 12 to instrument 20 via cable 14. Thus, control circuitry 16 of generator 12 may provide electrical power (via cable 14) having characteristics associated with the ultrasonic energy level/amplitude or type selected through control selector 30. Generator 12 may thus be operable to communicate different types or degrees of electrical power to ultrasonic transducer 26, in accordance with selections made by the operator via control selector 30. In particular, and by way of example only, generator 12 may increase the voltage and/or current of the applied signal to increase the longitudinal amplitude of the acoustic assembly. As a merely illustrative example, generator 12 may provide selectability between a “level 1” and a “level 5,” which may correspond with a blade 24 vibrational resonance amplitude of approximately 50 microns and approximately 90 microns, respectively. Various ways in which control circuitry 16 may be configured will be apparent to those skilled in the art in view of the teachings herein. It should also be understood that control selector 30 and activation switch 32 may be substituted with two or more activation switches 32. In some such versions, one activation switch 32 is operable to activate blade 24 at one power level/type while another activation switch 32 is operable to activate blade 24 at another power level/type, etc.

Conventionally, one or more buttons are provided that are operable in communication with the activation switches to activate a particular surgical mode with a corresponding power level/profile. In order to enable a small form factor, there is limited space to incorporate these and/or additional buttons for the operator to activate. Accordingly, and in accordance with the present disclosure, a sensor 38 is provided in communication with an arm 36 of the instrument 20. The sensor 38 is coupled with a measurement circuit 39 that is coupled to the activation switch 32. The sensor 38 measures a characteristic of the arm 36 while the end effector interacts with tissue of a patient, from which an amount of pressure experienced by the arm 36 is determined. Further discussion on operation of the arm 36 and sensor 38 is provided below.

In some alternative versions, control circuitry 16 is located within handpiece 22. For instance, in some such versions, generator 12 only communicates one type of electrical power (e.g., just one voltage and/or current available) to handpiece 22, and control circuitry 16 within handpiece 22 is operable to modify the electrical power (e.g., the voltage of the electrical power), in accordance with selections made by the operator via control selector 30, before the electrical power reaches ultrasonic transducer 26. Furthermore, generator 12 may be incorporated into handpiece 22 along with all other components of surgical system 10. For instance, one or more batteries (not shown) or other portable sources of power may be provided in handpiece 22. Still other suitable ways in which the components depicted in FIG. 1 may be rearranged or otherwise configured or modified will be apparent to those skilled in the art in view of the teachings herein.

### II. Overview of Exemplary Ultrasonic Surgical Instruments

The following examples relate to various non-exhaustive ways in which the technology disclosed herein may be employed in a physical system/instrument. It should be understood that the following examples are not intended to restrict the coverage of any claims that may be presented at any time in this application or in subsequent filings of this application. The following examples are being provided for nothing more than merely possible implementations to achieve the functionality described herein. It is contemplated that the various examples may be arranged and applied in numerous other ways. It is also contemplated that some variations may omit certain features referred to in the below examples.

More specifically, the following discussion relates to various exemplary components and configurations for instrument 20. It should be understood that the various examples of instrument 20 described below may be readily incorporated into a surgical system 10 as described above. It should also be understood that the various components and operability of instrument 20 described above may be readily incorporated into the exemplary versions of instruments 110, 210, and 310 described below. Various suitable ways in which the above and below teachings may be combined will be apparent to those skilled in the art in view of the teachings herein. It should also be understood that the below teachings may be readily combined with the various teachings of the references that are cited herein.

### A. Instrument with Sensor to Detect Inner Tube Pressure

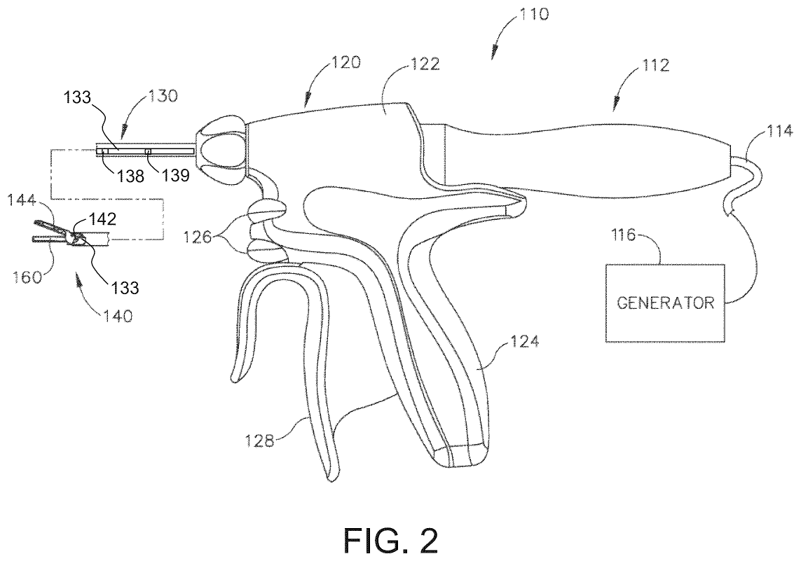

FIG. 2 illustrates an exemplary ultrasonic surgical instrument 110. At least part of instrument 110 may be constructed and operable in accordance with at least some of the teachings of U.S. Pat. Nos. 5,322,055; 5,873,873; 5,980,510; 6,325,811; 6,773,444; 6,783,524; 8,461,744; U.S. Pub. No. 2009/0105750; U.S. Pub. No. 2006/0079874; U.S. Pub. No. 2007/0191713; U.S. Pub. No. 2007/0282333; U.S. Pub. No. 2008/0200940; U.S. Pub. No. 2010/0069940; U.S. Pub. No. 2012/0112687; U.S. Pub. No. 2012/0116265; U.S. Pub. No. 2014/0005701; U.S. Pat. Pub. No. 2014/0114334; U.S. patent application Ser. No. 14/028,717; and/or U.S. Pat. App. No. 61/410,603. The disclosures of each of the foregoing patents, publications, and applications are incorporated by reference herein. As described therein and as will be described in greater detail below, instrument 110 is operable to cut tissue and seal or weld tissue substantially simultaneously. It should also be understood that instrument 110 may have various structural and functional similarities with the HARMONIC ACE® Ultrasonic Shears, the HARMONIC WAVE® Ultrasonic Shears, the HARMONIC FOCUS® Ultrasonic Shears, and/or the HARMONIC SYNERGY® Ultrasonic Blades. Furthermore, instrument 110 may have various structural and functional similarities with the devices taught in any of the other references that are cited and incorporated by reference herein.

To the extent that there is some degree of overlap between the teachings of the references cited herein, the HARMONIC ACE® Ultrasonic Shears, the HARMONIC WAVE® Ultrasonic Shears, the HARMONIC FOCUS® Ultrasonic Shears, and/or the HARMONIC SYNERGY® Ultrasonic Blades, and the following teachings relating to instrument 110, there is no intent for any of the description herein to be presumed as admitted prior art. Several teachings herein will in fact go beyond the scope of the teachings of the references cited herein and the HARMONIC ACE® Ultrasonic Shears, the HARMONIC WAVE® Ultrasonic Shears, the HARMONIC FOCUS® Ultrasonic Shears, and the HARMONIC SYNERGY® Ultrasonic Blades.

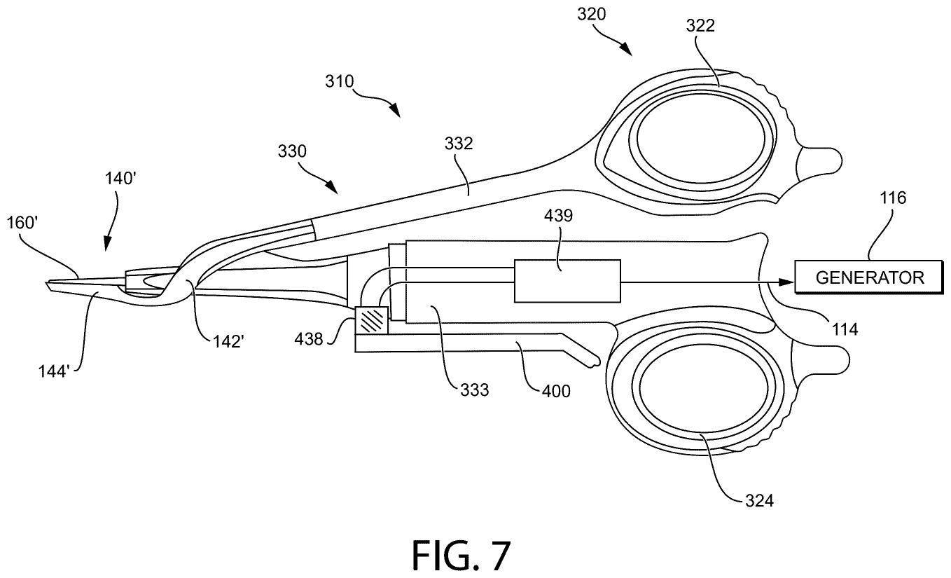

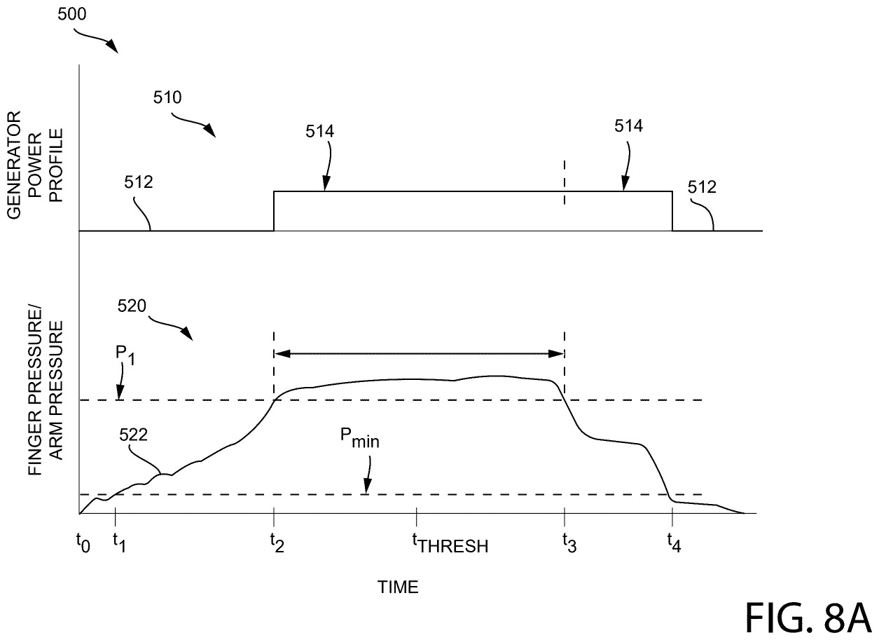

Instrument 110 of the present example comprises a handle assembly 120, a shaft assembly 130, and an end effector 140. Handle assembly 120 comprises a body 122 including a pistol grip 124 and a pair of buttons 126. Handle assembly 120 also includes a trigger 128 that is pivotable toward and away from pistol grip 124. It should be understood, however, that various other suitable configurations may be used, including, but not limited to, a scissor grip configuration (described in greater detail with regard to FIGS. 6 and 7). End effector 140 includes an ultrasonic blade 160 and a pivoting clamp arm 144. Ultrasonic blade 160 may be configured and operable just like ultrasonic blade 24 described above.

Clamp arm 144 is pivotably coupled with an inner tube 133 and an outer tube that form shaft assembly 130. Such an inner tube 133 and outer tube configuration may be provided in accordance with the teachings of various references that are cited herein. Clamp arm 144 is further coupled with trigger 128. Trigger 128 is operable to drive one of the tubes of shaft assembly 130 longitudinally while the other tube of shaft assembly 130 remains stationary. This relative longitudinal movement between the tubes of shaft assembly 130 provides pivotal movement of clamp arm 144. Clamp arm 144 is thus pivotable toward ultrasonic blade 160 in response to pivoting of trigger 128 toward pistol grip 124; and clamp arm 144 is pivotable away from ultrasonic blade 160 in response to pivoting of trigger 128 away from pistol grip 124. Clamp arm 144 is thereby operable to cooperate with ultrasonic blade 160 to grasp and release tissue; and clamp arm 144 is further operable to compress tissue against ultrasonic blade 160 to thereby enhance the communication of ultrasonic vibration from ultrasonic blade 160 to the tissue. Various suitable ways in which clamp arm 144 may be coupled with trigger 128 will be apparent to those skilled in the art in view of the teachings herein. In some versions, one or more resilient members are used to bias clamp arm 144 and/or trigger 128 to the open position shown in FIG. 2.

An ultrasonic transducer assembly 112 extends proximally from body 122 of handle assembly 120. Transducer assembly 112 may be configured and operable just like transducer 26 described above. Transducer assembly 112 is coupled with a generator 116 via a cable 114. It should be understood that transducer assembly 112 receives electrical power from generator 116 and converts that power into ultrasonic vibrations through piezoelectric principles. Generator 116 may be configured and operable like generator 12 described above. Generator 116 may thus include a power source and control module that is configured to provide a power profile to transducer assembly 112 that is particularly suited for the generation of ultrasonic vibrations through transducer assembly 112. It should also be understood that at least some of the functionality of generator 116 may be integrated into handle assembly 120, and that handle assembly 120 may even include a battery or other on-board power source such that cable 114 is omitted. Still other suitable forms that generator 116 may take, as well as various features and operabilities that generator 116 may provide, will be apparent to those skilled in the art in view of the teachings herein.

As shown in FIG. 2, by way of example, one of the buttons 126 may be associated with a “seal” mode, such that actuating the particular one of the buttons 126 only seals tissue, but does not cut tissue, when the tissue is being clamped between clamp arm 144 and blade 160. In particular, activation of a first one of the buttons 126 may cause vibration of ultrasonic blade 160 at a relatively low amplitude. Similarly, by way of further example, the other of the buttons 126 may be associated with a “cut and seal” mode such that actuating the particular one of the buttons 126 may seal and cut tissue when the tissue is being clamped between clamp arm 144 and blade 160. In particular, activation of a second one of the buttons 136 may cause vibration of ultrasonic blade 160 at a relatively high amplitude. Other suitable operational modes that may be associated with buttons 126 will be apparent to persons skilled in the art in view of the teachings herein.

While instruments similar to instrument 110 are effective for cutting and sealing tissue as described above, the buttons 126 provided thereon are large, which prevent smaller device geometry and also do not enable activation of the various surgical modes in a natural or ergonomic way, such as activation based on the grip pressure applied by the operator. Therefore, in addition to, or as an alternative to, the example depicted in FIG. 2 includes a sensor 138 in communication with a measurement circuit 139, which as discussed above, is connected to the activation switch. The sensor 138 is operably coupled to the inner tube 133 (also referred to herein as an “arm”) and, in conjunction with the measurement circuit 139, measures a characteristic thereof. In the present example, the sensor 138 measures a pressure and/or elongation of the inner tube 133 when the end effector 140 interacts with the tissue. The inner tube 133 is coupled to clamp arm 144 to cause its pivoting.

Due to the interrelatedness of their operation, the amount of grip (or finger) pressure applied by the operator to a portion of or the entire handle assembly 120, the pressure/elongation experienced by the inner tube 133, and the clamping pressure on the tissue have a predetermined relationship with one another when the end effector 140 interacts with the tissue. This predetermined relationship can be used to enable certain triggering events (e.g., a particular surgical mode) based on the applied finger pressure. In other words, the outputted signal from the sensor 138, which is measured by the measurement circuit 139, can be used to determine the finger pressure applied by the operator to the trigger 128. The finger pressure data can then be used in a variety of ways to serve as an activation trigger for one or more surgical modes (described in greater detail below).

In the present example, sensor 138 is embedded in the inner tube 133. However, sensor 138 may be coupled to or in communication with the inner tube 133 in other suitable ways as will be apparent to persons skilled in the art in view of the teachings herein. In the example shown, sensor 138 is configured to detect levels of elongation/compression of inner tube 133, from which the measurement circuit 139 may determine the pressure experienced by the inner tube 133 while the clamp arm 144 interacts with tissue.

In the present example, and the examples that follow in subsequent sections and figures, sensor 38, 138 comprises a strain gauge, with the small electrical resistive change created by the strain gauge being measurement by a suitable measurement circuit 139, such as a Wheatstone Bridge. In other examples, sensor 138 may comprise other types of sensors, such as an electroactive material, a piezoelectric sensor, a ferroelectric sensor, pressure sensitive layers of suitable materials such as graphene, and/or any other suitable kind(s) of sensors. Other suitable forms that sensor 138 may take will be apparent to persons skilled in the art in view of the teachings herein. In the example shown, there is only one sensor 138 of a single type. However, in other examples, there may be multiple sensors 138 of a single type; or multiple sensors 138 of multiple types.

The measurement circuit 139 communicates the sensed strain and/or pressure to generator 12, 116 via a wire or other component which is in electrical communication with generator 12, 116. In such examples, the measurement circuit 139 may communicate the sensed strain and/or pressure to generator 12, 116 wirelessly using known components and modalities. In some examples, the sensed strain may be communicated to a controller (not shown) that is located within instrument 110, which then converts the strain data to a pressure level associated with the strain data, which then communicates the pressure level to the generator 12, 116.

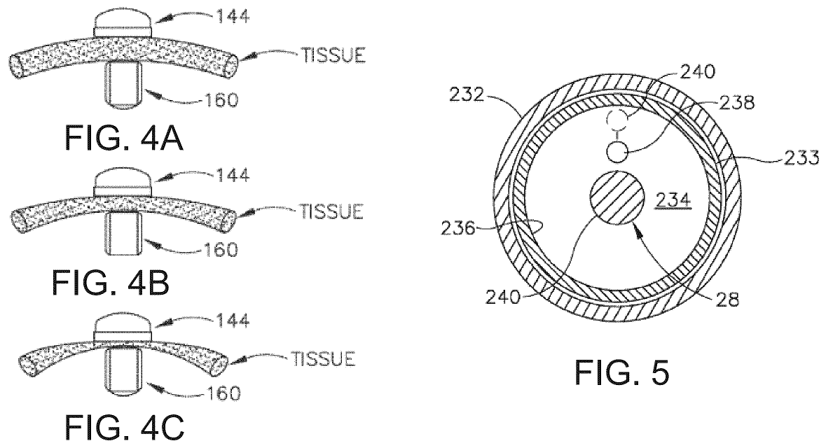

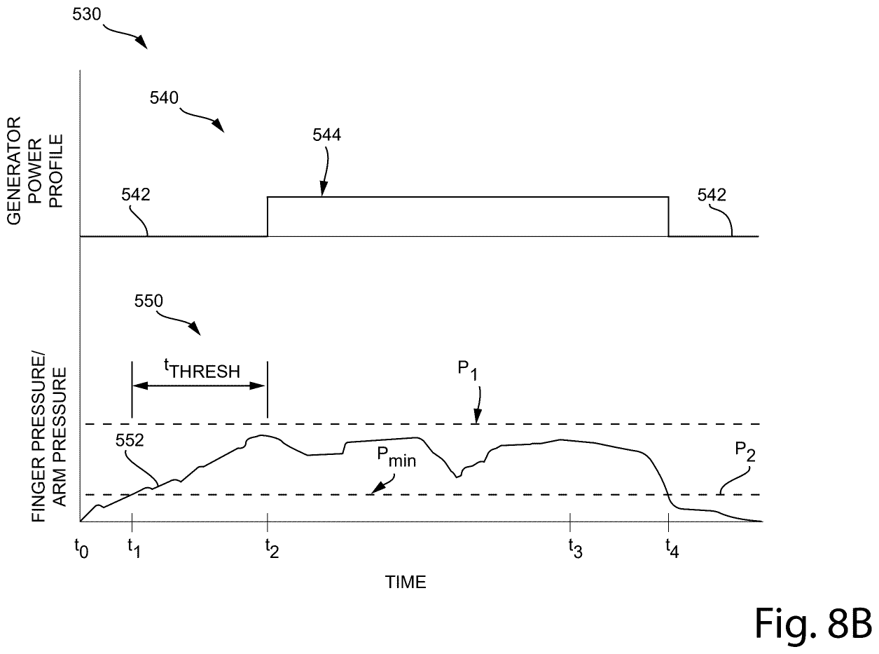

Generator 12, 116 is configured to deliver one or more power profiles to instrument 110. By way of example, as the tissue is being treated, the tissue state can change from an initial unsealed state (e.g., FIG. 4A) to a coagulated or partially sealed state (e.g., FIG. 4B), to a sealed state (e.g., FIG. 4C). At these various states, the operator may wish to enter a particular surgical mode (e.g., a “seal” mode). Rather than needing to rely upon buttons, in the instant case, one or more of these surgical modes can be activated by the operator applying a certain finger pressure profile to the trigger 128. The value of the pressure experienced by the inner tube 133 may thus serve as an informational proxy for the state of operator's finger pressure on the trigger 128. The details of activation of the one or more surgical modes/power profiles are described more fully in relation to FIGS. 8A-11.

In the example shown, generator 12, 116 or instrument 110 may communicate to the operator, via the output indicator 18, the state of the tissue based on various sensed parameters. For example, generator 12, 116 or instrument 110 may provide an indication to the operator that, based on the sensed parameter, the tissue is in the initial state, a partially sealed state, a sealed state, a cut and sealed state, or another tissue state that will be apparent to persons skilled in the art in view of the teachings herein. The indication to the operator may be visual, audio, physical (e.g., haptic), any other suitable indication modes, or combinations thereof.

Various other examples of features that may be used to monitor the finger pressure of an operator and adjust outputs of instrument 20, 110 are described in greater detail below.

### B. Instrument with Sensor to Detect Ultrasonic Blade Deflection

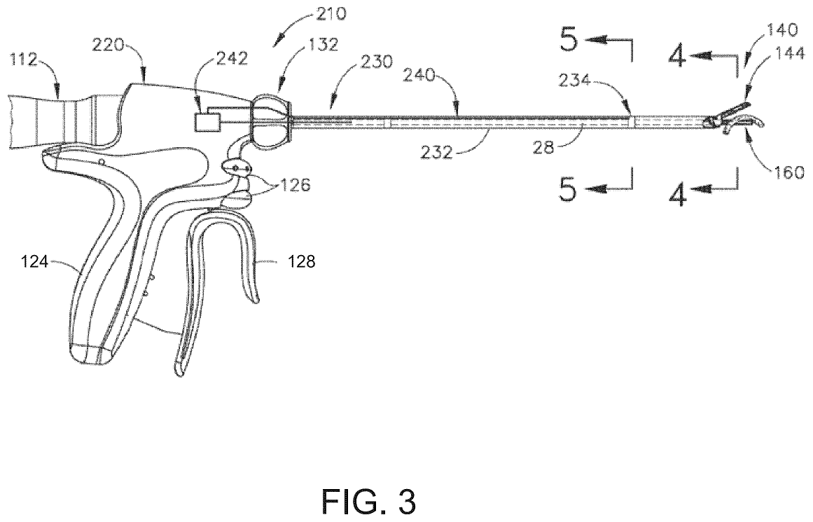

FIGS. 3-5 show an exemplary alternative instrument 210 that is substantially similar to instrument 110 described above. Therefore, identical or similar structures are labeled with like reference numerals without further explanation below. It should therefore be understood that instrument 210 may be readily incorporated into system 10 as a form of instrument 20. Instrument 210 of this example includes a handle assembly 220 that is just like handle assembly 120 described above. Handle assembly 220 is configured to receive an ultrasonic transducer 112. While not shown in FIG. 3, it should be understood that transducer 112 may be in communication with a generator 12, 116 via cable 14. A shaft assembly 230 extends distally from handle assembly 220. Shaft assembly 230 includes end effector 140 that is configured and operable substantially identically to end effector 140 described above. It should be understood, however, that shaft assembly 230 of this example is not limited to use with end effector 140. By way of example only, shaft assembly 230 may instead be readily combined with end effectors that are operable to apply electrosurgical energy to tissue, end effectors that are operable to apply staples to tissue, end effectors that are operable to apply sutures to tissue, end effectors that are operable to apply clips to tissue, etc.

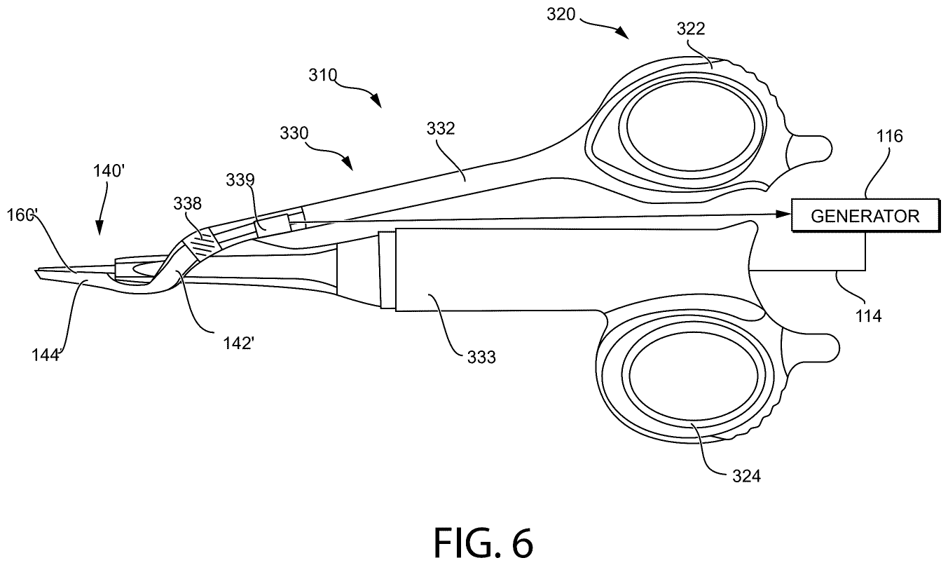

Shaft assembly 230 is similar to shaft assembly 130 such that it includes an outer tube 232, an inner tube 233 defining a lumen 236, a waveguide 28 coaxially disposed within tubes 232, 233, and a distal seal member 234 sealing off proximal portions of lumen 236. Trigger 128 is operable to drive one of the tubes of shaft assembly 230 longitudinally while the other tube of shaft assembly 130 remains stationary. This relative longitudinal movement between the tubes of shaft assembly 230 provides pivotal movement of clamp arm 144. Moreover, as shown best in FIG. 5, distal seal 234 includes a sensor 238 that is operably coupled to the distal seal 234. Because the inner aperture 240 of distal seal 234 is in touching contact with the outer portion (e.g., outer diameter) of waveguide 28 (also referred to herein as an “arm”), vibrations of waveguide 28 due to oscillation of waveguide 28 will be acoustically and mechanically transferred to distal seal 234 and, thus, sensor 238. In the present example, sensor 238 is embedded in distal seal 234. However, sensor 238 may be coupled to or in communication with distal seal 234 in other suitable ways as will be apparent to persons skilled in the art in view of the teachings herein. In the example shown, sensor 238 is configured to detect levels of lateral deflection of ultrasonic blade 160, from which the measurement circuit may determine the pressure experienced by the waveguide 28 while the blade 160 interacts with tissue. Shaft assembly 230 may be rotated using rotation knob 132.

Sensor 238 communicates the sensed deflection and/or pressure to the activation switch and/or the generator 12, 116 via wire 240 to contact ring 242, which is in electrical communication with generator 12, 116. While contact ring 242 is shown to provide electrical communication between wire 240 and generator 12, 116, in other examples, there may be suitable other structures that provide electrical communication between wire 240 and generator 12, 116. In some examples, wire 240 is omitted. In such examples, sensor 238 may communicate the sensed deflection and/or pressure to generator 12, 116 wirelessly using known components and modalities. In some examples, the sensed deflection may be communicated to a controller (not shown) that is located within instrument 210, which then converts the deflection data to a pressure level associated with the deflection data, which then communicates the pressure level to the generator 12, 116.

Due to the interrelatedness of their operation, the amount of grip (or finger) pressure applied by the operator to a portion of or the entire handle assembly 220, the pressure/deflection experienced by the waveguide 28, and the clamping pressure on the tissue have a predetermined relationship with one another when the end effector 140 interacts with the tissue. This predetermined relationship can be used to enable certain triggering events (e.g., a particular surgical mode) based on the applied finger pressure. In other words, the outputted signal from the sensor 238, which is measured by the measurement circuit, can be used to determine the finger pressure applied by the operator to the trigger 128. The finger pressure data can then be used in a variety of ways to serve as an activation trigger for one or more surgical modes (described in greater detail below).

The measurement circuit communicates the sensed strain and/or pressure to generator 12, 116 via a wire or other component which is in electrical communication with generator 12, 116. In such examples, the measurement circuit may communicate the sensed strain and/or pressure to generator 12, 116 wirelessly using known components and modalities. In some examples, the sensed strain may be communicated to a controller (not shown) that is located within instrument 210, which then converts the strain data to a pressure level associated with the strain data, which then communicates the pressure level to the generator 12, 116.

As in the example described in relation to FIG. 2, generator 12, 116 is configured to deliver one or more power profiles to instrument 110. By way of example, as the tissue is being treated, the tissue state can change from an initial unsealed state (e.g., FIG. 4A) to a coagulated or partially sealed state (e.g., FIG. 4B), to a sealed state (e.g., FIG. 4C). At these various states, the operator may wish to enter a particular surgical mode (e.g., a “seal” mode). Rather than needing to rely upon buttons, in the instant case, one or more of these surgical modes can be activated by the operator applying a certain finger pressure profile to the trigger 128. The value of the pressure experienced by the inner tube 133 may thus serve as an informational proxy for the state of operator's finger pressure on the trigger 128. The details of activation of the one or more surgical modes/power profiles are described more fully in relation to FIGS. 8A-11.

In the example shown, generator 12, 116 or instrument 210 may communicate to the operator, via the output indicator 18, the state of the tissue based on the sensed pressure level. For example, generator 12, 116 or instrument 210 may provide an indication to the operator that, based on the sensed pressure level, the tissue is in the initial state, a partially sealed state, a sealed state, a cut and sealed state, or another tissue state that will be apparent to persons skilled in the art in view of the teachings herein. The indication to the operator may be visual, audio, physical (e.g., haptic), any other suitable indication modes, or combinations thereof.

### C. Instrument Including Scissor Grips

FIG. 6 shows an exemplary alternative instrument 310 that is configured to operate similar to surgical instruments 110, 210, but with a scissor grip 320 as part of the handpiece 22 in place of a trigger. It should be understood that instrument 310 may be readily incorporated into system 10 as a form of instrument 20. As discussed in further detail below, instrument 310 is configured to sense different characteristics associated with an operator's finger pressure while clamping tissue and is further configured to deliver a particular amount of power to ultrasonic blade 160′ based on the sensed characteristics and/or other parameters.

More specifically, instrument 310 of the present example comprises a handle assembly 320, a shaft assembly 130, and an end effector 140′. Handle assembly 120 is embodied as a scissor grip and comprises a first finger grip 322 and a second finger grip 324. A shaft assembly 330 connects the handle assembly 320 with the end effector 140′ and comprises a first shaft 332 and a second shaft 333. End effector 140′ includes an ultrasonic blade 160′ and a pivoting clamp arm 144′ that operate similarly to the examples described above, but with the first finger grip 322 being operable to pivot the clamping arm 144′ about pivot point 140′ to interact with tissue at varying pressures. Ultrasonic blade 160′ may be configured and operable just like ultrasonic blade 24 described above.

As mentioned above, clamping arm 144′ is pivotably coupled with the first shaft 332 (i.e., the clamping arm 144′ pivots in unison with the first shaft), which is coupled to the first finger grip 322. Relative rotational movement between the finger grips 322, 324 provides pivotal movement of clamp arm 144'. Clamp arm 144′ is thus pivotable toward ultrasonic blade 160′ in response to pivoting of the first finger grip 322 towards the second finger grip 324; and clamp arm 144′ is pivotable away from ultrasonic blade 160′ in response to pivoting of the first finger grip 322 away from the second finger grip 324. Clamp arm 144′ is thereby operable to cooperate with ultrasonic blade 160′ to grasp and release tissue; and clamp arm 144′ is further operable to compress tissue against ultrasonic blade 160′ to thereby enhance the communication of ultrasonic vibration from ultrasonic blade 160′ to the tissue. In some versions, one or more resilient members are used to bias clamp arm 144′ and/or the first finger grip 322 to the open position.

An ultrasonic transducer assembly (not shown in this example) extends proximally from the second shaft 333 of the shaft assembly 330. Transducer assembly may be configured and operable just like transducer 26 described above. Transducer assembly is coupled with a generator 116 via a cable. It should be understood that transducer assembly receives electrical power from generator 116 and converts that power into ultrasonic vibrations through piezoelectric principles. Generator 116 may be configured and operable like generator 12 described above. Generator 116 may thus include a power source and control module that is configured to provide a power profile to transducer assembly that is particularly suited for the generation of ultrasonic vibrations through transducer assembly. It should also be understood that at least some of the functionality of generator 116 may be integrated into handle assembly 320, and that handle assembly 320 may even include a battery or other on-board power source such that cable 114 is omitted. Still other suitable forms that generator 116 may take, as well as various features and operabilities that generator 116 may provide, will be apparent to those skilled in the art in view of the teachings herein.

While instruments similar to instrument 310 are effective for cutting and sealing tissue, they do not enable activation of the various surgical modes in a natural or ergonomic way, such as activation based on the grip pressure applied by the operator. Rather, they include buttons, similar to the buttons 126 previously discussed. Therefore, in addition to or as an alternative to, a sensor 338 is provided in communication with a measurement circuit 339, which as discussed above in relation to the system overview of FIG. 1, is connected to an activation switch. The sensor 338 is operably coupled to the first shaft 332 (also referred to herein as an “arm”) close to the pivot point 142′ and, in conjunction with the measurement circuit 339, measures a characteristic of the first shaft 332. In the present example, the sensor 338 measures a level of lateral deflection and/or pressure of the first shaft 332 when the end effector 140′ interacts with the tissue.

Due to the interrelatedness of their operation, the amount of grip (or finger) pressure applied by the operator to the grips 322, 324 to a portion of or the entire handle assembly 120, the pressure/deflection experienced by the first shaft 332, and the clamping pressure on the tissue have a predetermined relationship with one another when the end effector 140′ interacts with the tissue. This predetermined relationship can be used to enable certain triggering events (e.g., a particular surgical mode) based on the applied finger pressure. In other words, the outputted signal from the sensor 338, which is measured by the measurement circuit 339, can be used to determine the finger pressure applied by the operator to the finger grips 322, 324. The finger pressure data can then be used in a variety of ways to serve as an activation trigger for one or more surgical modes (described in greater detail below).

In the present example, sensor 338 is embedded in first shaft 332. However, sensor 338 may be coupled to or in communication with the first shaft 332 in other suitable ways as will be apparent to persons skilled in the art in view of the teachings herein. In the example shown, sensor 338 is configured to detect levels of deflection/pressure of the first shaft 332, from which the measurement circuit 339 may determine the pressure experienced by the first shaft 332 while the clamp arm 144′ interacts with tissue.

The measurement circuit 339 communicates the sensed strain and/or pressure to generator 12, 116 via a wire or other component which is in electrical communication with generator 12, 116. In such examples, the measurement circuit 339 may communicate the sensed strain and/or pressure to generator 12, 116 wirelessly using known components and modalities. In some examples, the sensed strain may be communicated to a controller (not shown) that is located within instrument 310, which then converts the strain data to a pressure level associated with the strain data, which then communicates the pressure level to the generator 12, 116.

Generator 12, 116 is configured to deliver one or more power profiles to instrument 310. By way of example, as the tissue is being treated, the tissue state can change from an initial unsealed state (e.g., FIG. 4A) to a coagulated or partially sealed state (e.g., FIG. 4B), to a sealed state (e.g., FIG. 4C). At these various states, the operator may wish to enter a particular surgical mode (e.g., a “seal” mode). Rather than needing to rely upon buttons, in the instant case, one or more of these surgical modes can be activated by the operator applying a certain finger pressure profile to the finger grips 322, 324. The value of the pressure experienced by the first shaft 332 may thus serve as an informational proxy for the state of operator's finger pressure on the finger grips 322, 324. The details of activation of the one or more surgical modes/power profiles are described more fully in relation to FIGS. 8A-11.

In the example shown, generator 12, 116 or instrument 310 may communicate to the operator, via the output indicator 18, the state of the tissue based on various sensed parameters. For example, generator 12, 116 or instrument 310 may provide an indication to the operator that, based on the sensed parameter, the tissue is in the initial state, a partially sealed state, a sealed state, a cut and sealed state, or another tissue state that will be apparent to persons skilled in the art in view of the teachings herein. The indication to the operator may be visual, audio, physical (e.g., haptic), any other suitable indication modes, or combinations thereof.

### D. Instrument Including a Dedicated Lever

FIG. 7 shows an exemplary alternative instrument 310 that is configured to operate similar to surgical instruments 110, 210, and equivalently to previously described instrument in FIG. 6. Therefore, identical or similar structures are labeled with like reference numerals as that of FIG. 6 without further explanation below. It should be understood that instrument 310 may be readily incorporated into system 10 as a form of instrument 20. As discussed in further detail below, instrument 310 is configured to sense different characteristics associated with an operator's finger pressure while clamping tissue and is further configured to deliver a particular amount of power to ultrasonic blade 160′ based on the sensed characteristics and/or other parameters.

An ultrasonic transducer assembly (not shown in this example) extends proximally from the second shaft 333 of the shaft assembly 330. Transducer assembly may be configured and operable just like transducer 26 described above. Transducer assembly is coupled with a generator 116 via a cable. It should be understood that transducer assembly receives electrical power from generator 116 and converts that power into ultrasonic vibrations through piezoelectric principles. Generator 116 may be configured and operable like generator 12 described above. Generator 116 may thus include a power source and control module that is configured to provide a power profile to transducer assembly that is particularly suited for the generation of ultrasonic vibrations through transducer assembly. It should also be understood that at least some of the functionality of generator 116 may be integrated into handle assembly 320, and that handle assembly 320 may even include a battery or other on-board power source such that cable 114 is omitted. Still other suitable forms that generator 116 may take, as well as various features and operabilities that generator 116 may provide, will be apparent to those skilled in the art in view of the teachings herein.

Similarly to the previous example, a sensor 438 is provided in communication with a measurement circuit 439, which as discussed above in relation to the system overview of FIG. 1, is connected to an activation switch. However, in this example, the sensor 438 is operably coupled to a dedicated lever 400 (also referred to herein as an “arm”) and, in conjunction with the measurement circuit 439, measures a characteristic of the lever 400. In the present example, the sensor 438 measures a lateral deflection and/or pressure of the lever 400 when an operator presses on the lever 400 (e.g., with their index finger) while the operator holds the rest of the handle assembly 320 and the end effector 140′ interacts with the tissue. In the illustrated example, the lever 400 is depicted as coupled to the second shaft 333 and is free floating at the opposite end. Of course, in other examples, the lever 400 can be connected at other strategic locations on the instrument 310, such as, but not limited to, the first shaft 332, the first finger grip 322, or the second finger grip 324.

In this example, since the lever 400 functions separately from the clamping/cutting of the end effector 140′ (which is effected by operation of the handle assembly 320), the amount of grip (or finger) pressure applied by the operator to the lever 400 provides direct feedback regarding intentional operator input thereto. This direct feedback can be used to enable certain triggering events (e.g., a particular surgical mode) based on the applied finger pressure independent of the clamping pressure applied by the end effector 140′. In other words, the outputted signal from the sensor 438, which is measured by the measurement circuit 439, can be used to determine the finger pressure applied by the operator to the lever 400.

The finger pressure data can then be used in a variety of ways to serve as an activation trigger for one or more surgical modes (described in greater detail below). For example, the lever pressure could be used to enter a sealing mode and then, once the sealing mode is completed, the trigger 128 used to enable a cut mode after a successful seal. Additionally or alternatively, pressure from both the trigger 128 and the lever 400 could be used to enter a particular surgical mode (e.g., the sealing mode), whereas trigger only pressure 128 indicates/activates other surgical modes, such as a dissection or cauterization (or other) mode.

In the present example, sensor 438 is embedded in the lever 400. However, sensor 438 may be coupled to or in communication with the lever 400 in other suitable ways as will be apparent to persons skilled in the art in view of the teachings herein. In the example shown, sensor 438 is configured to detect levels of deflection/pressure of the lever 400, from which the measurement circuit 339 may determine the pressure experienced by the lever 400 when the operator presses on it.

The measurement circuit 339 communicates the sensed strain and/or pressure to generator 12, 116 via a wire or other component which is in electrical communication with generator 12, 116. In such examples, the measurement circuit 339 may communicate the sensed strain and/or pressure to generator 12, 116 wirelessly using known components and modalities. In some examples, the sensed strain may be communicated to a controller (not shown) that is located within instrument 310, which then converts the strain data to a pressure level associated with the strain data, which then communicates the pressure level to the generator 12, 116.

Generator 12, 116 is configured to deliver one or more power profiles to instrument 310. By way of example, as the tissue is being treated, the tissue state can change from an initial unsealed state (e.g., FIG. 4A) to a coagulated or partially sealed state (e.g., FIG. 4B), to a sealed state (e.g., FIG. 4C). At these various states, the operator may wish to enter a particular surgical mode (e.g., a “seal” mode). Rather than needing to rely upon buttons, in the instant case, one or more of these surgical modes can be activated by the operator applying a certain finger pressure profile to the lever 400, distinct from the finger pressure profile applied to the finger grips 322, 324. The value of the pressure experienced by the lever 400 may thus serve as an informational proxy for the state of operator's finger pressure on the lever 400 itself. The details of activation of the one or more surgical modes/power profiles are described more fully in relation to FIGS. 8A-11.

In the example shown, generator 12, 116 or instrument 310 may communicate to the operator, via the output indicator 18, the state of the tissue based on various sensed parameters. For example, generator 12, 116 or instrument 310 may provide an indication to the operator that, based on the sensed parameter, the tissue is in the initial state, a partially sealed state, a sealed state, a cut and sealed state, or another tissue state that will be apparent to persons skilled in the art in view of the teachings herein. The indication to the operator may be visual, audio, physical (e.g., haptic), any other suitable indication modes, or combinations thereof.

### IV. Exemplary Operation of a Generator and Surgical Instrument

Again making reference to FIG. 1, and as discussed above, the presently described system 10 employs a sensor 38 that measures a characteristic of the instrument 20 as it interacts with tissue (i.e., once it is clamping the tissue) to enable activation of a particular power profile. In this example, the generator 12 creates an ultrasonic electrical signal (also referred to as a drive signal, and delivered by an energy circuit) for driving the ultrasonic transducer 26. In some examples, the presently described power profile can comprise an ultrasonic electrical signal profile and its corresponding covert ultrasonic vibration energy output by the transducer.

In various forms, the generator 12 may comprise several separate functional elements, such as modules and/or blocks. Although certain modules and/or blocks may be described by way of example, it can be appreciated that a greater or lesser number of modules and/or blocks may be used and still fall within the scope of the forms. Further, although various forms may be described in terms of modules and/or blocks to facilitate description, such modules and/or blocks may be implemented by one or more hardware components, e.g., processors, Digital Signal Processors (DSPs), Programmable Logic Devices (PLDs), Application Specific Integrated Circuits (ASICs), circuits, registers and/or software components, e.g., programs, subroutines, logic and/or combinations of hardware and software components.

In some examples, the generator 12 may comprise one or more embedded applications implemented as firmware, software, hardware, or any combination thereof. The generator 12 may comprise various executable modules such as software, programs, data, drivers, application program interfaces (APIs), and so forth. The firmware may be stored in nonvolatile memory (NVM), such as in bit-masked read-only memory (ROM) or flash memory. In various implementations, storing the firmware in ROM may preserve flash memory. The NVM may comprise other types of memory including, for example, programmable ROM (PROM), erasable programmable ROM (EPROM), electrically erasable programmable ROM (EEPROM), or battery backed random-access memory (RAM) such as dynamic RAM (DRAM), Double-Data-Rate DRAM (DDRAM), and/or synchronous DRAM (SDRAM).

In some examples, the generator 12 comprises a hardware component implemented as a processor for executing program instructions for monitoring various measurable characteristics of the ultrasonic surgical instrument 10 (FIG. 1) and generating a step function output signal for driving the ultrasonic transducer in cutting and/or coagulation operating modes. It will be appreciated by those skilled in the art that the generator 12 may comprise additional or fewer components and only a simplified version of the generator 12 are described herein for conciseness and clarity. In various forms, the hardware component may be implemented as a DSP, PLD, ASIC, circuits, and/or registers. In one form, the processor may be configured to store and execute computer software program instructions to generate the step function output signals for driving various components of the ultrasonic surgical instrument 10, such as a transducer, an end effector, and/or a blade 24.

In some examples, under control of one or more software program routines, the processor executes the methods in accordance with the described forms to generate a step function formed by a stepwise waveform of drive signals comprising current (I), voltage (V), and/or frequency (f) for various time intervals or periods (T). The stepwise waveforms of the drive signals may be generated by forming a piecewise linear combination of constant functions over a plurality of time intervals created by stepping the generator 12 drive signals, e.g., output drive current (I), voltage (V), and/or frequency (f). The time intervals or periods (T) may be predetermined (e.g., fixed and/or programmed by the user) or may be variable. Variable time intervals may be defined. The ultrasonic drive signals generated by the generator 12 include, without limitation, ultrasonic drive signals capable of exciting the ultrasonic transducer 26 in various vibratory modes such as, for example, the primary longitudinal mode and harmonics thereof as well flexural and torsional vibratory modes.

In some examples, the executable modules comprise one or more step function algorithm(s) (e.g., the algorithms depicted in logic flow charts 900, 1100) stored in memory that when executed causes the processor to generate a step function formed by a stepwise waveform of drive signals comprising current (I), voltage (V), and/or frequency (f) for various time intervals or periods (T). The stepwise waveforms of the drive signals may be generated by forming a piecewise linear combination of constant functions over two or more time intervals created by stepping the generator's output drive current (I), voltage (V), and/or frequency (f). The drive signals may be generated either for predetermined fixed time intervals or periods (T) of time or variable time intervals or periods of time in accordance with the one or more stepped output algorithm(s). Under control of the processor, the generator 12 steps (e.g., increment or decrement) the current (I), voltage (V), and/or frequency (f) up or down at a particular resolution for a predetermined period (T) or until a predetermined condition is detected, such as a change in a monitored characteristic (e.g., transducer impedance, tissue impedance). The steps can change in programmed increments or decrements. If other steps are desired, the generator 12 can increase or decrease the step adaptively based on measured system characteristics.

In operation, the user can program the operation of the generator 12 using the input device located on the front panel of the generator console. The input device may comprise any suitable device that generates signals that can be applied to the processor to control the operation of the generator 12. In various forms, the input device includes buttons, switches, thumbwheels, keyboard, keypad, touch screen monitor, pointing device, remote connection to a general purpose or dedicated computer. In other forms, the input device may comprise a suitable user interface. Accordingly, by way of the input device, the user can set or program the current (I), voltage (V), frequency (f), and/or period (T) for programming the step function output of the generator 12. The processor then displays the selected power level by sending a signal online to an output indicator 18 (FIG. 1).

More particularly, and in accordance with the disclosed technology, operation of the generator 12 can be activated based on the sensed characteristic of an arm 36 of the surgical instrument 10. Upon triggering of a pressure, time, or pressure/time threshold based on the sensed characteristic of the arm 36, a specific executable module associated with a particular power profile/surgical mode (e.g., a “seal” mode, as discussed above) is executed, and is discussed in greater detail below. In some examples, the module to be executed upon being triggered is selectable by the operator who will be using the surgical instrument 10.

In various forms, the output indicator 18 may provide visual, audible, and/or tactile feedback to the surgeon to indicate the status of a surgical procedure, such as, for example, when tissue cutting and coagulating is complete based on a measured characteristic of the ultrasonic surgical instrument, e.g., transducer impedance, tissue impedance, or other measurements as subsequently described. By way of example, and not limitation, visual feedback comprises any type of visual indication device including incandescent lamps or light emitting diodes (LEDs), graphical user interface, display, analog indicator, digital indicator, bar graph display, digital alphanumeric display. By way of example, and not limitation, audible feedback comprises any type of buzzer, computer generated tone, computerized speech, voice user interface (VUI) to interact with computers through a voice/speech platform. By way of example, and not limitation, tactile feedback comprises any type of vibratory feedback provided through the instrument handle assembly (e.g., handle assemblies 120, 220, 320).

In normal use, the instrument 10 can operate with a constant non-algorithmic profile (e.g., exemplary use cases include dissection, cauterization for an RF device, etc.). It is noted, however, that even “constant” profiles can be dynamic, to a certain degree, based on tissue load experiences (which can affect the harmonic levels). In the context of the present disclosure, a constant non-algorithmic profile refers to when substantially the same electrical power is applied within the respective power profile. The following examples describe the activation of a particular executable module (e.g., a specific power profile associated with a surgical mode) on the basis of the grip/finger pressure of an operator which, as discussed above, can be determined by a sensor 38 measuring a characteristic of an arm 36 of the instrument 10. It should be understood that the following examples are not intended to restrict the coverage of any claims that may be presented at any time in this application or in subsequent filings of this application.

### A. Surgical Mode Activated with a Time and Pressure Threshold

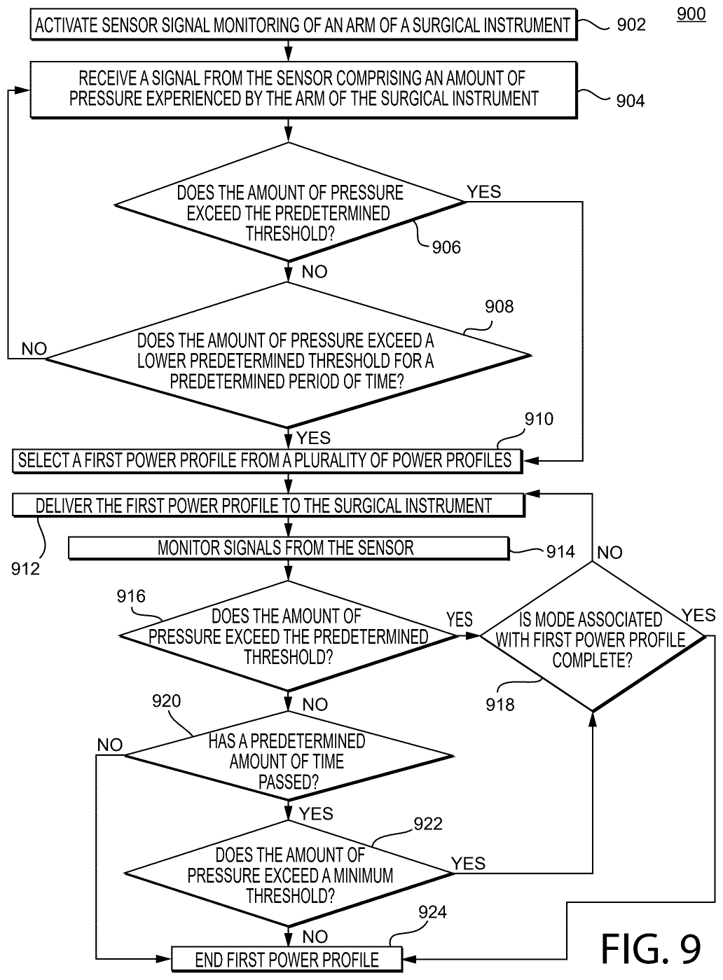

FIG. 8A depicts a graph 500 of finger pressure 520 and generator power 510 profiles over time using an instrument of the system of FIG. 1, in accordance with the disclosed technology. FIG. 9 depicts a logic flow diagram for selecting a power profile corresponding to a tissue algorithm that may be implemented by a generator, in accordance with the disclosed technology. The presently disclosed technology includes algorithms that, when a certain parameter or set of parameters is met, trigger the processor to deliver a predetermined power profile, via the drive signal, to the ultrasonic transducer 26, which converts that signal and delivers (in the present example) ultrasonic vibrational energy to the blade 24 (via the waveguide 28).

The logic flow diagram 900 in FIG. 9 depicts one form/method of selecting and delivering a predetermined power profile (e.g., the power profile shown in FIG. 8A) to the blade 24 on the basis of an operator's finger pressure on a portion of the surgical instrument 10. More specifically, and in relation to FIGS. 8A and 9, the disclosed technology includes a system/method that utilizes time and pressure thresholds to activate and maintain a particular surgical mode. By way of example, as discussed above, this surgical mode can include, but is not limited to, a sealing mode of the generator 12.

Making reference to FIGS. 1, 8A and 9 in conjunction with one another, the sensor signal monitoring of an arm 36 of a surgical instrument 10 is activated 902. The monitoring may be implemented by a measurement circuit 39, directly by the generator 12 (e.g., by a processor within the generator 12), and/or by any other appropriate circuits/processors. In some examples, a first-stage processing (digitizing) circuit is provided inside the surgical instrument 10, which sends a digitized representation of the pressure to the generator through digital communications. The generator processor then reads the finger pressure and compares to time/pressure thresholds for activation and profile determination (discussed in greater detail below). In some examples, the monitoring is activated 902 after it is determined that tissue is being clamped by the end effector of the surgical instrument 10.

For example, an operator's finger pressure 522 on the surgical instrument 10 (e.g., the trigger 128, handle assembly 320, or lever 400) may vary over time as depicted in FIG. 8A, beginning at a starting time t₀. At a first time t₁, the operator's finger pressure crosses over a minimum pressure threshold Pₘᵢₙ. At a second time t₁, the operator's finger pressure crosses over a first pressure threshold P₁. At a third time t₃, the operator's finger pressure goes below the first pressure threshold P₁ but above the minimum pressure threshold Pₘᵢₙ. At a fourth time t₄, the operator's finger pressure goes below the minimum pressure threshold Pₘᵢₙ.

Over time, signals are received 904 from the sensor 38. The signals comprise a characteristic of the arm 36 (e.g., the strain thereon while the end effector interacts with/clamps the tissue), from which an amount of pressure experience by the arm 36 can be determined. As discussed above, depending upon the arm 36 utilized for measurement in relation to the rest of the instrument 10, the pressure experienced by the arm 36 has a corresponding mechanical relationship/correlation with the finger pressure applied by the operator. A minimum activation threshold (e.g., Pₘᵢₙ or P₁) can be correlated through testing to equate to a certain clamping pressure, thus guaranteeing that a sufficient tissue clamp pressure is reached before activation is permitted. Therefore, FIG. 8A's depiction of finger pressure also correlates to the pressure experienced by the arm 36 in the presently described system.

On the basis of the signals, the generator 12 (or other appropriate circuitry/processor) determines 906 whether the amount of pressure exceeds a predetermined pressure threshold. In the case of FIG. 8A, the first pressure threshold P₁ corresponds to the predetermined pressure threshold. As shown in the upper graph of FIG. 8A, prior to the first pressure threshold P₁ being exceeded, no power profile 512 is delivered to the surgical instrument 10 from the generator 12.

It is noted that, in this time and pressure threshold example, the determining step 908 depicted in FIG. 9 can be omitted. It is discussed in greater detail in section IV. C. below.

In response to the first pressure threshold P₁ being exceeded, a surgical mode with a corresponding first power profile 514 is selected 912 from a group of one or more power profiles and continuously delivered 514 to the blade 24 of the surgical instrument 10.

After the first pressure threshold P₁ has been exceeded, and as the first power profile 514 is continuously delivered to the blade 24, the generator 12 (or other appropriate circuitry/processor) continues to monitor 914 signals from the sensor 38. As signals from the sensor 38 are received, they are evaluated to determine 916 whether the amount of the pressure experienced by the arm 36 exceeds the first pressure threshold P₁.

If the amount of pressure does exceed the first pressure threshold P₁, the generator 12 (or other appropriate circuitry/processor) determines 918 whether the surgical mode corresponding to the first power profile 514 has been completed. By way of example, the generator 12 can determine whether the activated sealing mode has been completed. If the surgical mode/cycle is not complete, the logic flow diagram 900 loops back to block 912 and continues/sustains delivery of the first power profile 514. If the surgical mode/cycle is complete, delivery of the first power profile 514 is ended/stopped 924.

If the amount of pressure does not exceed the first pressure threshold P₁, the generator 12 (or other appropriate circuitry/processor) determines 920 whether a predetermined amount of time has passed. In relation to FIG. 8A, this corresponds to tₜₕᵣₑₛₕ. At this point, the generator 12 maintains delivery of the specified surgical mode/first power profile 514 as long as at least the minimum pressure threshold Pₘᵢₙ (which is lower than the first pressure threshold P₁) is maintained. This enables the operator to reduce the amount of finger/grip pressure they apply to the surgical instrument 10 without causing the surgical mode to automatically/mistakenly/prematurely be disabled, which would otherwise cause the mode to be cut short before it is completed.

More specifically, and with continued reference to FIGS. 8A and 9, if the signal received indicates that the first pressure threshold P₁ is no longer being exceeded, the generator 12 (or other appropriate circuitry/processor) determines 920 whether the predetermined amount of time tₜₕᵣₑₛₕ has passed. If the predetermined amount of time tₜₕᵣₑₛₕ has not passed, delivery of the first power profile 514 is ended/stopped 924. Additionally or alternatively, a “warning” (e.g., in the form of visual or audio feedback) can be output to the indicator 18 to indicate to the operator that the surgical mode has been or will be prematurely ended due to the predetermined amount of time tₜₕᵣₑₛₕ not elapsing.

If the signal received indicates that the first pressure threshold P₁ is no longer being exceeded, and the generator 12 (or other appropriate circuitry/processor) determines 920 that the predetermined amount of time tₜₕᵣₑₛₕ has passed, the generator 12 determines 922 whether the amount of pressure exceeds the minimum pressure threshold Pₘᵢₙ.

If the amount of pressure does exceed the minimum pressure threshold Pₘᵢₙ, the generator 12 determines 918 whether the surgical mode corresponding to the first power profile 514 has been completed. If the surgical mode/cycle is not complete, the logic flow diagram 900 loops back to block 912 and continues/sustained delivery of the first power profile 514. If the surgical mode/cycle is complete, delivery of the first power profile 514 is ended/stopped 924. For example, as depicted in FIG. 8A, at the third time t ₃ (and up to the fourth time t₄), the arm pressure falls below the first pressure threshold P₁, but stays above the minimum pressure threshold Pₘᵢₙ. Thus, delivery of the first power profile 514 is continued/sustained.

If the pressure does not exceed the minimum pressure threshold Pₘᵢₙ, delivery of the first power profile 514 is ended/stopped 924. This is exemplified at the fourth time t₄ where the arm pressure dips below the minimum pressure threshold Pₘᵢₙ. Additionally or alternatively, a “warning” (e.g., in the form of visual or audio feedback) can be output to the indicator 18 to indicate to the operator that the surgical mode has been or will be prematurely ended due to the minimum pressure threshold Pₘᵢₙ no longer being exceeded.

By utilizing the foregoing logic flow, the present system enables continuous delivery of a certain surgical mode without requiring application of a constant and/or very high finger pressure by the operator while providing certain safeguards against unintentional activation and maintaining of the surgical mode. However, as those skilled in the art will appreciate, monitoring of the arm 38 may be used in other ways without departing from the spirit and scope of the present disclosure, such as the examples described below.

### B. Surgical Mode Activated with a Pressure Threshold

Reference is again made to FIGS. 8A and 9 with respect to a modification of the above example. In this example, no time threshold tₜₕᵣₑₛₕ is employed. In other words, the logic flow described in section IV. A. above can be identically performed as described, but with the omission of the determination block 920 where it is determined whether a predetermined amount of time has passed when the generator 12 decides whether to continue delivery of the first power profile 514 to the blade 24 or to end/stop delivery of it. Therefore, in this example, the exceeding of the first pressure threshold P₁ causes delivery of the first power profile to the surgical instrument 10, and this surgical mode can be continued/sustained either until either (i) it has completed or (ii) the finger pressure applied by the operator falls below the minimum pressure threshold Pₘᵢₙ required to keep the surgical mode activated.

For example, by default, the instrument 10 can be configured to activate in a “cut” mode (e.g., dissection, etc.). Upon the operator pressing harder, a “seal mode” is enabled. As long as a minimum required pressure (informed, at least in part, by a minimum pressure required to achieve sufficient clamp pressure for good tissue sealing) is maintained, the seal cycle will continue to completion (even if finger pressure falls below initial activation pressure threshold).

### C. Surgical Mode Activated with a Time Threshold

FIG. 8B depicts another graph 530 of finger pressure 550 and generator power 540 profiles over time using an instrument 10 of the system of FIG. 1, in accordance with the disclosed technology.

Reference is now made to FIG. 8B and again to FIG. 9 with respect to a modification of the above example. In this example, a pressure threshold P₁ is not specifically employed to activate a certain surgical mode. Rather, a time threshold tₜₕᵣₑₛₕ is employed in conjunction with the exceeding of a second pressure threshold P₂ in order to activate a power profile 542. In some examples, P₂ can be lower than P_{1.} In others, P₂ can be the same as P₁. As shown in FIG. 8B, the operator's finger pressure crosses the second pressure threshold P₂ at a first time t₁, but no power 542 is delivered from the generator 12. Rather, the threshold time t ₜₕᵣₑₛₕ must elapse and, at a second time t₂ after the second pressure threshold P₂ has been maintained, the power profile 544 associated with a surgical mode is activated. Similar to the above-described examples, at the fourth time t₄, when the finger pressure drops below a minimum pressure threshold P_{min,} delivery of the power profile 544 is ended.

In other words, the logic flow diagram 900 described in section IV. A. above can be identically performed as described, but with the omission of the determination blocks 906 and 920 and use of determination block 908. Therefore, in this example, after receiving 904 a signal from the sensor, it is determined 908 whether the pressure exceeds a pressure threshold P₂ for a predetermined period of time tₜₕᵣₑₛₕ.

If the pressure does exceed this threshold P₂ for the predetermined period of time tₜₕᵣₑₛₕ, the logic flow diagram 900 continues as described above to blocks 910 and onwards, with the exception of block 920 being optionally eliminable in terms of determining whether to continue delivery of the power profile 544 or not.

If the time threshold tₜₕᵣₑₛₕ and/or pressure threshold P₂ has not been met, no power profile is delivered to the blade 24 and the logic flow diagram 900 loops back to block 904 to continue to monitor for signals from the sensor 36.

### D. Different Surgical Modes Dynamically Activated and Scaled with Varying Grip Pressure

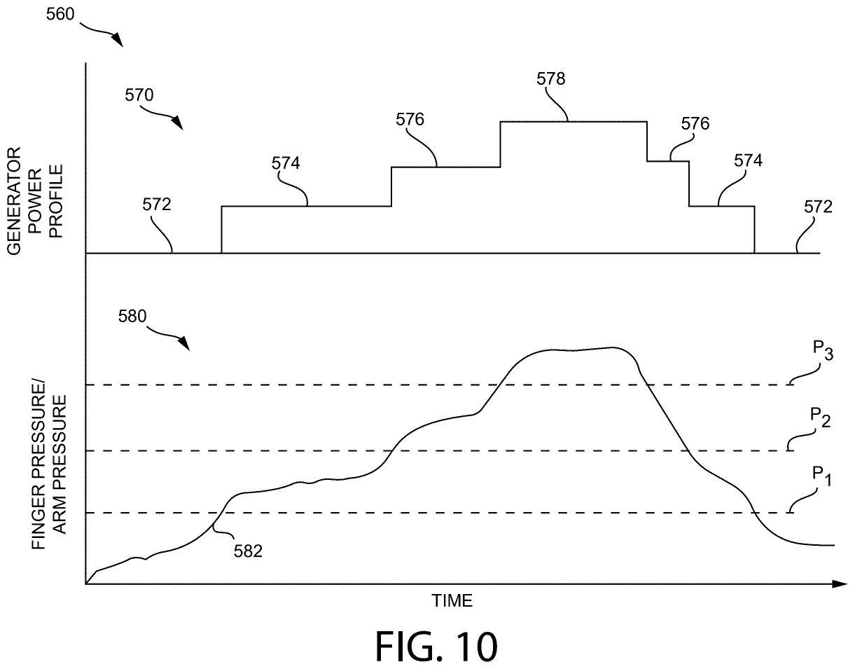

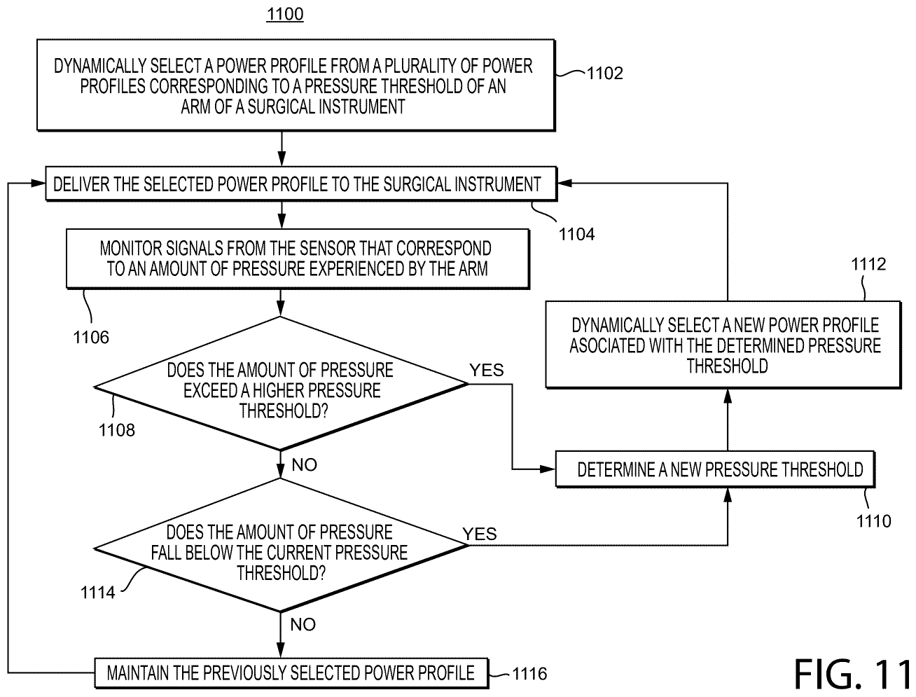

FIG. 10 depicts another graph 560 of finger pressure 580 and generator power 570 profiles over time using an instrument of the system of FIG. 1, in accordance with the disclosed technology. FIG. 11 depicts another logic flow diagram 1100 for selecting a power profile, from a plurality of discrete power profiles (e.g., power profiles 572, 574, 576, 578), corresponding to a tissue algorithm that may be implemented by a generator, in accordance with the disclosed technology. In the presently described example, the power profile 570 can be correlated to operator pressure once above a minimum/activation pressure threshold P₁. This example can operate as a stand-alone algorithm/method, or in conjunction with the previously described methods as a sub-routine in place of or supplementing steps 910 and 912 (e.g., dynamically scaling the power at different thresholds in the examples described with respect to FIGS. 8A-9).

Reference is now made to FIG. 10 and to FIG. 11. While clamping tissue, and upon application of finger pressure over a pressure threshold (e.g., a first pressure threshold P₁), a surgical mode with a corresponding power profile 574 is dynamically selected 1102 from a plurality of power profiles and delivered 1104 to the blade 24 based on a determined amount of pressure experience by the arm 36. This process can similar to that of the foregoing examples where a non-zero minimum pressure threshold Pₘᵢₙ is needed to activate a power profile, or it can be implemented where a minimum threshold of pressure is zero.

In some examples, similar to the examples described above, a time threshold can be (but does not necessarily need to be) implemented in the dynamic selection step to ensure that the power profile is being intentionally activated and/or dynamically scaled up or down by the surgeon. By way of example, one power profile 576 can be delivered to the end effector in response to the amount of pressure falling within a first pressure range (e.g., above P2). If the amount of pressure experienced by the arm 36 falls to within a second pressure range (between P1 and P2), less than the first pressure range, for a period of time, another power profile 574, less than the previous power profile 576, can be dynamically scaled and delivered to the end effector. By way of example, this period of time threshold can be 50-400 milliseconds.

After the first pressure threshold P₁ has been exceeded, and as the first power profile 574 is continuously delivered to the blade 24, the generator 12 (or other appropriate circuitry/processor) continues to monitor 1106 signals from the sensor 38. As signals from the sensor 38 are received, they are evaluated to determine 1108 whether the amount of the pressure experienced by the arm 36 exceeds a higher pressure threshold P₂ and/or determine 1114 whether the amount of the pressure experienced by the arm 36 falls below the first pressure threshold P₁. In other words, the generator 12 monitors to determine whether the amount of pressure is within the same pressure threshold it previously was or if it falls within a new (higher or lower) pressure threshold.

If the amount of pressure does exceed the second pressure threshold P₂, a new pressure threshold P₂ is determined/set 1110, and a new power profile 576 that corresponds to the new pressure threshold P₂ is dynamically selected 1112. At this point, the logic flow diagram 1100 loops back to block 1104 and delivers the new selected power profile 576 to the blade 24. As seen in FIG. 10, this process enables the power profiles 574, 576, 578 to dynamically scale with increasing or decreasing pressure 582 applied by the operator.

If the amount of pressure does not exceed the second pressure threshold P₂, the generator 12 (or other appropriate circuitry/processor) determines 1114 whether the amount of pressure previously determined falls below the current pressure threshold (in the present example, the first pressure threshold P₁).

If the pressure does not fall below the first pressure threshold P₁, the power profile 574 corresponding to the first pressure threshold P₁ is maintained 1116 (i.e., a new pressure threshold and new power profile is not selected). At this point, the logic flow diagram 1100 loops back to block 1104 and delivers the same selected power profile 574 to the blade 24.

If the pressure does fall below the first pressure threshold P₁, the power profile delivery can be stopped 572. Alternatively, in other examples, the minimum pressure threshold for a non-zero power profile to be delivered can be approximately zero. In other words, reference number 572 can also correspond to a power profile 572 that is associated with an arm pressure that is at or near zero, which allows further customization of how the arm pressure feature can be used by the surgeon or another operator.

In a scenario where, for example, power profile 578 is being delivered, and it is determined that the third pressure threshold P₃ is no longer being exceeded but the second pressure threshold P₂ is, a new lower pressure threshold is determined 1110, and the power profile 576 corresponding to that lower threshold is dynamically selected 1112 and scaled/delivered 1104 to the blade.

It is noted that, in this example and as shown in FIG. 10, each pressure range (e.g., between P₁ and P₂, and between P₂ and P₃) includes a predetermined range of pressures therein. Moreover, each power profile associated with a respective pressure range can be configured to maintain a uniform power profile (creating stepped power profiles that dynamically adjust based on the sensed pressure). In this way, each discrete power profile maintains a uniform power until the power profile is dynamically selected and scaled based on the arm pressure falling within a different pressure range.

By utilizing the foregoing logic flow 1100, the present system enables continuous delivery of a certain surgical mode as well as the ability to enter higher or lower power surgical modes depending upon the finger pressure applied by the operator.

### V. Exemplary Supplements and Combinations to the Foregoing

As those skilled in the art will appreciate, the foregoing implementations/uses of the surgical instrument may be modified or supplemented in various ways. For example, a warning tone or visual indicator may be enabled to provide an indication the pressure is close to sufficient for activation, thus alerting the operator that additional pressure will result in activation of a particular surgical mode (which may or may not be desired). An audio tone or other form of alert can be generated to indicate the surgical mode has been entered, with another tone/indicator being generated at completion of the cycle. A changed pitch in tone or change in light color/visual indication can be used to indicate an increase or decrease in power output from the generator 12 as the operator presses harder or releases pressure. A solid tone could be used to indicate dissection/quick bursts (like monopolar tones), whereas cyclical tones could be used to indicate a sealing mode output (like Hemo tones).

Moreover, it is noted that, while the power profiles in the foregoing examples are primarily described in the context of providing uniform/constant power that is less or more than a power of a different power profile, some of the surgical modes usable with the presently disclosed technology do not necessarily generate a constant power level. Rather, they are dependent on time and the tissue's real-time response. Sometimes, the same electrical power may be applied within a respective power profile, but the shape of the power delivery is different to cause different tissue effects (i.e., there are variable power limits throughout the mode rather than a “fixed” power limit). For example, a “first power profile” could be left unchanged throughout a thirty second activation, whereas a “second power profile” associated with a higher pressure threshold may, at times, be less (i.e., lower supplied electrical power) and, at times, be more (i.e., higher supplied electrical power) and, at other times, be equal (i.e., same supplied electrical power) to the first power profile throughout the said thirty second activation. In other words, the second power profile in this example is different from the first power profile, but not necessarily consistently more or less than the first power profile throughout its run time.

The following clauses relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

The disclosed technology described herein can be further understood according to the following clauses:
Clause 1. A surgical system comprising: a surgical instrument comprising: a handle assembly configured to be grasped by one or more hands of an operator; a shaft assembly connected to the handle assembly; an arm; an end effector connected to the shaft assembly and configured to interact with tissue; and a sensor configured to sense a characteristic of the arm, from which an amount of pressure experienced by the arm while the end effector interacts with the tissue is determined; and a generator connected to the surgical instrument and configured to: dynamically scale a power profile to deliver to the end effector from a plurality of power profiles, the power profile being selected based on the amount of pressure falling within one of a plurality of pressure ranges; deliver a first power profile to the end effector in response to the amount of pressure falling within a first pressure range; and scale to and deliver a second power profile, less than the first power profile, to the end effector in response to the amount of pressure falling within a second pressure range, less than the first pressure range, for a first period of time.
Clause 2. The surgical system of clause 1, whereby the determined amount of pressure experienced by the arm comprises a predetermined relationship with an amount of finger pressure applied by the operator to a portion of the handle assembly.
Clause 3. The surgical system of clause 2, wherein the portion of the handle assembly comprises a trigger configured to have the finger pressure applied thereto.
Clause 4. The surgical system of clause 2, wherein the portion of the handle assembly comprises a first finger grip and a second finger grip configured to have the finger pressure applied thereto.
Clause 5. The surgical system of clause 2, wherein the portion of the handle assembly comprises the arm, the arm being configured to have the finger pressure applied directly thereto.
Clause 6. The surgical system of clause 1, wherein the characteristic is a level of deflection of the arm.
Clause 7. The surgical system of clause 1, wherein the end effector comprises a clamp arm and a blade, the shaft assembly comprises a first shaft and a second shaft drivable longitudinally relative to the first shaft to pivot the clamp arm relative to the blade, and the arm comprises the second shaft.
Clause 8. The surgical system of clause 1, wherein the end effector comprises a clamp arm and a blade, the shaft assembly comprises a first shaft, a second shaft drivable longitudinally relative to the first shaft to pivot the clamp arm relative to the blade, and a waveguide, and the arm comprises the waveguide.
Clause 9. The surgical system of clause 1, wherein the end effector comprises a clamp arm and a blade, the shaft assembly comprises a first shaft and a second shaft pivotal relative to the first shaft to pivot the clamp arm relative to the blade, and the arm comprises the first shaft.
Clause 10. The surgical system of clause 1, wherein, in response to the determined amount of pressure falling below a minimum pressure threshold, the generator is configured to (i) cease delivery of the first or second power profile or (ii) activate an audio or visual indicator.
Clause 11. The surgical system of clause 1, wherein the generator is configured to: continue delivery of the first power profile to the end effector; monitor the amount of pressure over time; in response to the amount of pressure falling below the first pressure range before a first predetermined period of time has elapsed, stop delivery of the power profile to the end effector; in response to the amount of pressure exceeding the first pressure threshold until at least the first predetermined period of time has elapsed: determine whether the amount of pressure exceeds a minimum threshold; in response to the amount of pressure exceeding the minimum threshold, continue delivery of the first power profile to the end effector; and in response to the amount of pressure falling below the minimum threshold, stop delivery of the first power profile to the end effector.
Clause 12. The surgical system of clause 1, wherein the end effector comprises an ultrasonic blade, and the power profile comprises ultrasonic power.
Clause 13. The surgical system of clause 1, wherein the power profile comprises bipolar radiofrequency power.
Clause 14. The surgical system of clause 1, wherein the power profile is associated with an operation mode of the generator, and the surgical system further comprises: an audio or visual indicator that is indicative of a beginning or a completion of the operation mode.
Clause 15. The surgical system of clause 1, wherein the generator is configured to deliver a third power profile in response to the amount of pressure being approximately zero.
Clause 16. The surgical system of clause 1, wherein the first period of time is greater than 50-400 milliseconds.
Clause 17. The surgical system of clause 1, wherein each pressure range of the plurality of pressure thresholds has a predetermined range of pressures therein, and wherein each power profile of the plurality of power profiles maintains a uniform power.
Clause 18. The surgical system of clause 1, wherein the second pressure range includes a zero amount of pressure sensed, and second power profile includes a non-zero power delivered to the end effector.
Clause 19. A surgical system comprising: a surgical instrument comprising: a handle assembly configured to be grasped by one or more hands of an operator; a shaft assembly connected to the handle assembly; an arm; an end effector connected to the shaft assembly and configured to interact with tissue; and a sensor configured to sense a characteristic of the arm, from which an amount of pressure experienced by the arm while the end effector interacts with the tissue is determined; and a generator connected to the surgical instrument and configured to deliver, to the end effector, a power profile dynamically selected from a plurality of power profiles based on the determined amount of pressure experienced by the arm.
Clause 20. The surgical system of clause 19, whereby the determined amount of pressure experienced by the arm comprises a predetermined relationship with an amount of finger pressure applied by the operator to a portion of the handle assembly.
Clause 21. The surgical system of clause 19, wherein the characteristic is a level of deflection of the arm.
Clause 22. The surgical system of clause 19 wherein the plurality of profiles comprises: a first power profile associated with a first pressure threshold; and a second power profile associated with a second pressure threshold higher than the first pressure threshold, the first power profile or the second power profile being respectively dynamically selected based on the determined amount of pressure experienced by the arm being equal to or exceeding the first pressure threshold or the second pressure threshold.
Clause 23. The surgical system of clause 22, wherein the first pressure threshold is approximately zero.
Clause 24. A surgical instrument comprising: a handle assembly configured to be grasped by one or more hands of an operator; a shaft assembly connected to the handle assembly; an arm; and an end effector connected to the shaft assembly and configured to interact with tissue in response to actuation of the arm, the surgical instrument being configured to deliver a dynamically scaled amount of power delivered to the end effector based on an amount of pressure experienced by the arm while the end effector interacts with the tissue, the amount of power being selected from a plurality of discrete power profiles, each of the discrete power profiles being selected based on the amount of pressure falling within one of a plurality of pressure ranges, wherein each pressure range of the plurality of pressure ranges has a predetermined range of pressures therein, and wherein each power profile of the discrete power profiles maintains a uniform power until the amount of power delivered to the end effector is rescaled.
Clause 25. A generator comprising a control circuit configured to: receive a first signal from a surgical instrument (904), wherein the first signal comprises an amount of pressure experienced by an arm of a surgical instrument while the surgical instrument interacts with a tissue; dynamically select, from a plurality of discrete power profiles, and based on the determined amount of pressure experienced by the arm, a first power profile, wherein each of the discrete power profiles is selected based on the amount of pressure falling within one of a plurality of pressure ranges, with each pressure range of the plurality of pressure ranges having a predetermined range of pressures therein; and deliver, via an energy circuit, the first power profile of the plurality of profiles to the surgical instrument.
Clause 26. The generator of clause 25, wherein a sealing mode for sealing the tissue comprises the first power profile.
Clause 27. A surgical system comprising: a surgical instrument comprising: a handle assembly configured to be grasped by one or more hands of an operator; a shaft assembly connected to the handle assembly and comprising an arm; an end effector connected to the shaft assembly and configured to interact with tissue; and a sensor configured to sense a characteristic of the arm, from which an amount of pressure experienced by the arm while the end effector interacts with the tissue is determined; and a generator connected to the surgical instrument and configured to: monitor the amount of pressure over time; in response to the amount of pressure exceeding a first pressure threshold, deliver a power profile to the end effector at a first time; determine whether a predetermined period of time has elapsed since the first time; in response to the amount of pressure falling below the first pressure threshold before the predetermined period of time has elapsed, stop delivery of the power profile to the end effector; and in response to the amount of pressure exceeding the first pressure threshold for the predetermined period of time: determine whether the amount of pressure exceeds a minimum threshold; in response to the amount of pressure exceeding the minimum threshold, continue delivery of the power profile to the end effector; and in response to the amount of pressure falling below the minimum threshold, stop delivery of the power profile to the end effector.

The embodiments described above are cited by way of example, and the present invention is not limited by what has been particularly shown and described hereinabove. Rather, the scope of the invention includes both combinations and sub combinations of the various features described and illustrated hereinabove, as well as variations and modifications thereof which would occur to persons skilled in the art upon reading the foregoing description and which are not disclosed in the prior art.

1. A surgical system comprising:
a surgical instrument comprising:
a handle assembly configured to be grasped by one or more hands of an operator;
a shaft assembly connected to the handle assembly;
an arm;
an end effector connected to the shaft assembly and configured to interact with tissue; and
a sensor configured to sense a characteristic of the arm, from which an amount of pressure experienced by the arm while the end effector interacts with the tissue is determined; and
a generator connected to the surgical instrument and configured to:
dynamically scale a power profile to deliver to the end effector from a plurality of power profiles, the power profile being selected based on the amount of pressure falling within one of a plurality of pressure ranges;
deliver a first power profile to the end effector in response to the amount of pressure falling within a first pressure range; and
scale to and deliver a second power profile, less than the first power profile, to the end effector in response to the amount of pressure falling within a second pressure range, less than the first pressure range, for a first period of time.

2. The surgical system of claim 1, whereby the determined amount of pressure experienced by the arm comprises a predetermined relationship with an amount of finger pressure applied by the operator to a portion of the handle assembly.

3. The surgical system of claim 2, wherein the portion of the handle assembly comprises a trigger configured to have the finger pressure applied thereto.

4. The surgical system of claim 2, wherein the portion of the handle assembly comprises a first finger grip and a second finger grip configured to have the finger pressure applied thereto.

5. The surgical system of claim 2, wherein the portion of the handle assembly comprises the arm, the arm being configured to have the finger pressure applied directly thereto.

6. The surgical system of claim 1, wherein the characteristic is a level of deflection of the arm.

7. The surgical system of claim 1, wherein
the end effector comprises a clamp arm and a blade,
the shaft assembly comprises a first shaft and a second shaft drivable longitudinally relative to the first shaft to pivot the clamp arm relative to the blade, and
the arm comprises the second shaft.

8. The surgical system of claim 1, wherein
the end effector comprises a clamp arm and a blade,
the shaft assembly comprises a first shaft, a second shaft drivable longitudinally relative to the first shaft to pivot the clamp arm relative to the blade, and a waveguide, and
the arm comprises the waveguide.

9. The surgical system of claim 1, wherein
the end effector comprises a clamp arm and a blade,
the shaft assembly comprises a first shaft and a second shaft pivotal relative to the first shaft to pivot the clamp arm relative to the blade, and
the arm comprises the first shaft.

10. The surgical system of claim 1, wherein
the end effector comprises an ultrasonic blade, and
the power profile comprises ultrasonic power.

11. The surgical system of claim 1, wherein the generator is configured to deliver a third power profile in response to the amount of pressure being approximately zero.

12. The surgical system of claim 1, wherein the first period of time is greater than 50-400 milliseconds.

13. The surgical system of claim 1, wherein each pressure range of the plurality of pressure thresholds has a predetermined range of pressures therein, and wherein each power profile of the plurality of power profiles maintains a uniform power.

14. The surgical system of claim 1, wherein the second pressure range includes a zero amount of pressure sensed, and second power profile includes a non-zero power delivered to the end effector.

15. A surgical system comprising:
a surgical instrument comprising:
a handle assembly configured to be grasped by one or more hands of an operator;
a shaft assembly connected to the handle assembly;
an arm;
an end effector connected to the shaft assembly and configured to interact with tissue; and
a sensor configured to sense a characteristic of the arm, from which an amount of pressure experienced by the arm while the end effector interacts with the tissue is determined; and
a generator connected to the surgical instrument and configured to deliver, to the end effector, a power profile dynamically selected from a plurality of power profiles based on the determined amount of pressure experienced by the arm.

16. The surgical system of claim 15, whereby the determined amount of pressure experienced by the arm comprises a predetermined relationship with an amount of finger pressure applied by the operator to a portion of the handle assembly.

17. The surgical system of claim 15, wherein the characteristic is a level of deflection of the arm.

18. The surgical system of claim 15, wherein the plurality of profiles comprises:
a first power profile associated with a first pressure threshold; and
a second power profile associated with a second pressure threshold higher than the first pressure threshold,
the first power profile or the second power profile being respectively dynamically selected based on the determined amount of pressure experienced by the arm being equal to or exceeding the first pressure threshold or the second pressure threshold.

19. The surgical system of claim 18, wherein the first pressure threshold is approximately zero.

20. A surgical instrument comprising:
a handle assembly configured to be grasped by one or more hands of an operator;
a shaft assembly connected to the handle assembly;
an arm; and
an end effector connected to the shaft assembly and configured to interact with tissue in response to actuation of the arm,
the surgical instrument being configured to deliver a dynamically scaled amount of power delivered to the end effector based on an amount of pressure experienced by the arm while the end effector interacts with the tissue, the amount of power being selected from a plurality of discrete power profiles, each of the discrete power profiles being selected based on the amount of pressure falling within one of a plurality of pressure ranges,
wherein each pressure range of the plurality of pressure ranges has a predetermined range of pressures therein, and wherein each power profile of the discrete power profiles maintains a uniform power until the amount of power delivered to the end effector is rescaled.
